(19) United States
(12) Patent Application Publication
(10) Pub. No.: US 2006/0135422 A1
(43) Pub. Date: Jun. 22, 2006

(54) Use of angiotensin receptor blockers (ARBs) to treat diseases associated with excess ACE

(76) Inventor: David W. Moskowitz, St. Louis, MO (US)

Correspondence Address: Sonnenschein Nath & Rosenthal, P. O. Box 061080, Wacker Drive Station, Sears Tower, Chicago, IL, 60606-1080, US

(21) Appl. No.: 10/820,479

(22) Filed: Mar. 31, 2004

Related U.S. Application Data
(60) Provisional application No. 60/463,437, filed on Apr. 17, 2003.
(60) Provisional application No. 60/465,908, filed on Apr. 25, 2003.
(60) Provisional application No. 60/473,262, filed on May 27, 2003.
(60) Provisional application No. 60/477,387, filed on Jun. 11, 2003.
(60) Provisional application No. 60/482,553, filed on Jun. 26, 2003.
(60) Provisional application No. 60/500,933, filed on Sep. 8, 2003.

(51) Int. Cl. A61K 38/54

(52) U.S. Cl. 514/12

(57) ABSTRACT

Disclosed are methods of treating neuropsychiatric diseases, neuromuscular diseases, viral infections, neurodegenerative diseases and ricin poisoning using angiotensin II receptor blockers.

This application claims priority to U.S. Ser. No. 60/458,853, filed Mar. 31, 2003; U.S. Ser. No. 60/463,437 filed Apr. 17, 2003; U.S. Ser. No. 60/465,908 filed Apr. 25, 2003; U.S. Ser. No. 60/473,262 filed May 27, 2003; U.S. Ser. No. 60/477,387 filed Jun. 11, 2003; U.S. Ser. No. 60/482,553 filed Jun. 26, 2003; U.S. Ser. No. 60/500,933 filed Sep. 8, 2003; U.S. Provisional Application titled “Use of Angiotensin II Blockade Against All Viral Diseases: The Universal Viral Antidote,” filed December 2003;

### BACKGROUND

PCT/US02/25001, entitled “Methods and Compositions for Treating Diseases Associated with Excesses in ACE,” describes the discivery that numerous common diseases, congestive heart failure due to hypertension (HTN) or non-insulin dependent diabetes mellitus (type II diabetes mellitus)(NIDDM), atherosclerotic peripheral vascular disease due to HTN or NIDDM, and chronic obstuctive pulmonary disease are associated with the ACE D/D genotype and thus respond to treatment with ACE inhibitors. PCT/US03/33501 describes the treatment of several additional chronic diseases with ACE inhibitors. Both of the above identified applications are incoporated herein by reference.

Genomic epidemiologic data, increasingly supported by clinical outcomes results, strongly suggest that overactivity of angiotensin I-converting enzyme (ACE) may underlie most age-related diseases. Angiotensin II, the main product of ACE, is a pleiotropic hormone, capable of serving as a neurotransmitter, growth factor, angiogenesis factor, vasoconstrictor, pro-thrombotic agent, and cytokine. So it is perhaps not surprising that the ACE D/D genotype is associated with several major psychiatric diseases, most cancers except prostate cancer (where the D/D genotype is actually protective), most cardiovascular diseases, most autoimmune diseases, and even infectious diseases like tuberculosis and HIV. In a preliminary study, angiotensin II blockade appeared to hasten recovery from West Nile virus encephalitis; it just before the “Cambrian Explosion” in the number of species. The ancestral, unduplicated form of ACE is still expressed during the terminal differentiation of human spermatocytes, suggesting a critical role in reproduction. The crystal structure of testicular ACE (tACE) was recently published. Computer modeling suggests that tACE may be activated by both mechanical forces and reducing agents. The duplicated form of ACE (somatic ACE, sACE) is expressed in areas of high fluid flow. sACE may auto-dimerize via a novel protein motif, the “disulfide zipper.” The sACE dimer is predicted to have higher catalytic efficiency and redox resistance than tACE.

### INTRODUCTION

It is a tenet of modern biochemistry that form dictates function. This disclosure therefore begins with conjecture, based on available evidence, about the structure of somatic ACE (sACE), which has yet to be solved crystallographically. Angiotensin II, the major product of ACE, activates protein kinase C and the AP-1 transcription factor, which are very widely used in signal transduction. Redox- and mechanical activation of ACE could explain the enzyme's central role in pathophysiology. Overactivity of ACE appears to drive most common age-related diseases in vertebrates. Since there are a number of ACE inhibitors and angiotensin II receptor Blockers (ARB's) already available, this may be excellent news for public health.

Testicular ACE: Redox- and Mechanosensor?

Testicular ACE (tACE), the ancestral form of the molecule with a single active site, is a type I membrane protein with seven highly conserved cysteines. Of these, six are linked by disulfide bridges in a nearest neighbor, aabbcc, pattern [1]. The crystal structure of tACE in the presence of chloride was recently published [2].

The active site of tACE resembles a small box [2,3]. Zinc-dependent peptide bond hydrolysis appears to occur within a hole in the floor of the box which can accommodate nothing larger than a dipeptide [2]. A flow-sensitive “flap” occluding the two active sites of sACE was predicted [4]; a mobile “lid” composed of two alpha helices not hydrogenbonded to substrate (lisinopril) or the rest of the protein was observed in tACE [2]. This lid may allow the enzyme to function as a mechanosensor in areas of turbulent flow [4].

In tACE, cystines link the short beta sheets β1 (C¹⁸³) to β2 (C¹⁸⁹), β4 (C³⁸³) to β5 (C⁴⁰¹), and the alpha helix α17 (C⁵⁶⁹) to the 3₁₀ helix H7 (C⁵⁸¹) (amino acid numbering as in the tACE precursor, Swiss-Prot P22966, with structural motifs numbered as in [2]). In sACE, the corresponding cystines of the N-terminal domain link C¹⁵⁷ to C¹⁶⁵, C³⁵⁹ to C³⁷⁷, and C⁵⁴⁵ to C⁵⁵⁷ (numbering as in sACE precursor, Swiss-Prot P12821). C⁵⁰³ is a free sulfhydryl group. In the C-terminal domain, the corresponding disulfide bonds are between C⁷⁵⁷ and C⁷⁶³, C⁹⁵⁷ and C⁹⁷⁵, and C¹¹⁴³ and C¹¹⁵⁵. C¹¹⁰¹ is free. C⁵⁰³ does not appear to engage in disulfide bonding with C¹¹⁰¹ [1]. In the model proposed below, C⁵⁰³ and C¹¹⁰¹ fail to interact because they are located on opposite sides of a sphere.

To emphasize the molecule's underlying homology, in this review C-terminal domain cysteines will be referred to according to their N-terminal homologues, e.g. C³⁵⁹ and C³⁷⁷ instead of C⁹⁵⁷ and C⁹⁷⁵. For conceptual simplicity, amino acids will be numbered according to their position in sACE rather than tACE.

The cystine linking domains β4 β5 (C³⁵⁹—S—S—C³⁷⁷) is close enough to the active site (FIG. 1) that β4 and β5 create a wall on one side of the active site. This side wall binds to substrate with at least one hydrogen bond [2]. Cystine C³⁵⁹—S—S—C³⁷⁷ may act as a latch whose reduction may allow the side wall to swing open (FIG. 2). This possibility is suggested by substitution of a single cysteine by alanine, and calculation of the resultant structure of tACE using GROMOS96, a free energy-minimization program (FIG. 3, [5]). Thus, substrate should be free to enter, and product to leave, the active site after reduction of C³⁵⁹—S—S—C³⁷⁷. In silico “reduction” of either of the two other cystines (C¹⁸³—S—S—C¹⁸⁹ or C⁵⁶⁹—S—S—C⁵⁸¹) separately did not alter the structure of tACE, but “reduction” of any two cystines dramatically altered the enzyme's tertiary structure (data not shown). The enzyme is therefore hypothesized to be activated by limited reduction, e.g. by glucose or other sugars, hypoxia, low pH, or homocysteine.

Experimentally, however, tACE is resistant to even exhaustive reduction. A 900-fold molar excess of dithiothreitol inhibited tACE's activity by only 78% [1]. The enzyme appears to be more sensitive to oxidation [6]. Besides “locking up” the side wall and preventing substrate entry, oxidation may also lead to formation of cysteine sulfuric acid [7], which cannot form a disulfide bond.

From the discussion above, it appears that tACE could already serve as a redox- and mechanosensor. tACE has a lid which could be opened by turbulent flow, and a key disulfide bridge at C³⁸³—S—S—C⁴⁰¹ which could be reduced by hypoxia, low pH, etc. Access to the active site, and hence activity, may be highest when both the lid and the side wall are open, i.e. for enzyme exposed to turbulent flow under reducing conditions.

Normoxia would keep the C³⁸³—S—S—C⁴⁰¹ cystine in its oxidized state, with the side wall shut. Perhaps only a swimming spermatid could mechanically activate tACE protruding from its plasma membrane [8]. This would have the benefit of reserving scarce fuel [9] for the sole use of motile sperm. Motion-activated tACE would generate a local, extracellular concentration gradient of angiotensin II for autocrine stimulation of the spermatid. Angiotensin II type 1 (AT1) receptors located in the tail of the spermatid [10] could efficiently transduce the mechanical signal to mitochondria located in the neck of the spermatid [11]. The result would be to increase fuel and oxygen consumption, energy production, and forward motility [10,12].

Once a spermatid reaches the more acidic and hypoxic region of the cervix and has to slow down [13], redox sensing rather than mechanosensing might take over to activate tACE. After fertilization, redox activation of tACE located in the surface membrane of the zygote, gently floating along the hypoxic and acidic Fallopian tube, could result in continued local production of angiotensin II. Angiotensin II may trigger the post-fertilization zygote to switch from slow meiotic to rapid mitotic divisions [14]. Redox activation of zygotic tACE with continued angiotensin II production may stimulate angiogensis and uterine smooth muscle cell proliferation after implantation [15].

Structure of Somatic Ace

Somatic ACE (sACE), the duplicated form of the enzyme with two active sites, is a type I membrane protein like tACE. sACE anchored in the plasma membrane of endothelial cells projects minimally into the vascular lumen [16]. Besides endothelial cells, sACE is also present on epithelia exposed to high flow, such as the brush border membrane of the kidney proximal tubule, jejunal microvilli, and the choroid plexus. Interestingly, these tissues share the unusual ability to undergo hypertrophy. Angiotensin II, the product of sACE, helps initiate compensatory renal growth [16].

The structure of the N-terminal domain of sACE is still unknown, although the C-terminal domain is expected to be identical to tACE. The N-terminal domain seems to be more resistant to denaturation by heat or thiols than the C-terminal domain [17-19], suggesting that there might be differences in the tertiary structure of the two domains.

Hydrophobic ACE inhibitors like ramipril inhibit nearly 100% of serum ACE activity, whereas hydrophilic ACE inhibitors like enalapril inhibit only about 50%. This 2:1 ratio suggests that hydrophobic ACE inhibitors may bind to both active sites of sACE, whereas hydrophilic ACE inhibitors bind only to one [20]. Clinically, hydrophobic ACE inhibitors appear to be more effective than hydrophilic ones [20-22] (Table 1). For example, quinapril was more effective than ramipril at delaying the progression of chronic renal failure [20], and ramipril more effective at lowering pulmonary hypertension than enlapril [20,22]. Maximal inhibition of tissue ACE appears to be an appropriate clinical goal [25,26].

More hydrophobic ACE inhibitors have a somewhat higher binding affinity. For example, captopril has an IC₅₀ of 9.7 nM vs. 1.7 nM for zofenoprilat; enalaprilat has an IC₅₀ of 2.8 nM vs. only 0.67 nM for ramiprilat [27]. The “off time” for hydrophobic ACE inhibitors like quinapril and ramipril is 24 hr vs. only 4 hr for a hydrophilic ACE inhibitor like enalapril [28-30]. This suggests that hydrophobic ACE inhibitors are able to gain access to a different kind of active site than hydrophilic ACE inhibitors. The simplest explanation for all these data is that the N-terminal active site is more hydrophobic than the C-terminal active site. Hydrophobic ACE inhibitors can gain access to both active sites of sACE, whereas hydrophilic ACE inhibitors bind only to the C-terminal domain active site. Having to displace a hydrophobic autoinhibitory tripeptide (FQP) from the N-terminal domain active site might explain why this active site can be accessed only by hydrophobic ACE inhibitors at the doses used clinically [4,31].

Strong sequence homology nevertheless suggests that the two domains are at least somewhat similar in structure. If so, then the two domains may auto-dimerize via a novel motif, a “disulfide zipper” (FIG. 4). The three cystines from each domain can easily be interposed. Indeed, there appears to be a “tongue-in-groove” fit along a relatively flat surface at the bottom of each domain of the holoenzyme (FIG. 4b).

The six disulfides might even form an extended electron transport chain (FIG. 5), analogous to an iron-sulfur cluster without iron atoms [32]. Perhaps reducing equivalents interact with the free, conserved cysteine accessible to solvent (C⁵⁰³ or C¹¹⁰¹), present on opposite sides of the autodimer (FIG. 4b). Electrons may tunnel from the surface of the homodimer through to the disulfide zipper located along the interior seam of the autodimer [33].

Reduction of one domain's cystine “latch” (C³⁵⁹—S—S—C³⁷⁷ or C⁹⁵⁷—S—S—C⁹⁷⁵, the equivalent cystine in the C-terminal domain) might occur at the expense of the other domain's cystine through a disulfide isomerase reaction (FIG. 6). Relative to tACE, sACE might gain an additional catalytic mechanism involving two “swinging gates” (FIG. 7). A limiting amount of reductant could perhaps lead to a reciprocating or “ping-pong” mechanism whereby one cystine “latch” opened only after the other cystine “latch” closed. In theory, this could be set in motion by a single reducing equivalent.

No such “perpetual motion” mechanism could apply to a single domain enzyme like tACE. A single reducing equivalent (2 electrons or hydride ion) could start the side walls of both active sites of sACE flapping (FIG. 7), whereas it would lead to only a single, non-repeated action by the side wall of tACE (FIGS. 2,3).

When ACE underwent gene duplication, a single-shot pistol may have become a machine gun. The effect may have been an enormous gain in sensitivity to reducing equivalents by sACE as compared to tACE. A single reducing equivalent would result in production of far more angiotensin II by sACE than by tACE, for a huge systems gain [34]. Furthermore, the two “latches” (cystines) participating in the “swinging gate” mechanism would be inaccessible to solvent, since they would be part of the disulfide per at the interior of the autodimer (FIG. 4b).

The overall effect of gene duplication might therefore be a tremendous increase in catalytic efficiency for sACE relative to tACE, as well as increased resistance to redox inactivation [6]. This latter feature would be especially advantageous for an ectoenzyme on the surface of macrophages whose product, angiotensin II, stimulates the respiratory oxidative burst via protein kinase C [35].

The Secretase

An endo-proteolytic ectoenzyme (“secretase”) cleaves sACE from its membrane-bound stalk to release soluble sACE [36,37]. The activity of the secretase is stimulated by protein kinase C (PKC) [38-40]. The soluble form of sACE does not appear to contribute to disease [41], although it may help maintain systemic blood pressure [42,43]. Given that the major product of sACE, angiotensin II, also stimulates PKC, the secretase may thus participate in a negative feedback loop to decrease tissue ACE activity. The secretase may have an additional role, however.

The specific activities of secretase-deficient forms of sACE, including tACE, secretase-cleaved soluble sACE, detergent-solubilized sACE, and recombinant sACE are all similar [44-46], but may not reflect the enzyme's activity in situ. Pulmonary sACE has over 30 times the specific activity of sACE in other organs [47], and contains the secretase [37].

Soluble sACE may not remain in the same autodimeric structure as postulated above for membrane-bound, secretase-associated sACE. Once cleaved by the secretase, soluble sACE may no longer be able to function as a “reciprocating enzyme” (FIGS. 6,7). The soluble enzyme may retain an active N- or C-terminal domain with a single reduced cystine “latch,” but without the ability to regenerate activity in the other domain. This would occur if the disulfide zipper came apart, preventing disulfide isomerase activity. This might explain the striking negative cooperativity of soluble sACE observed by Kost and her colleagues [19].

The secretase which releases sACE from the plasma membrane might help hold the two domains of sACE together to form an autodimer (FIG. 4b). The C-domain of sACE is glycosylated, although somewhat less so than the N-terminal domain (7 vs. 10 Asn's). The carbohydrate residues bound to the N-terminal domain appear to promote autodimerization and binding to the secretase [48,49]. In other words, the secretase may function as a chaperone to bring the two domains of sACE together. Binding of sACE by the secretase might explain why antibodies directed against membrane-bound sACE recognized only the N-terminal domain, and not the C-terminal domain [50].

sACE and Physiology

Angiotensin II and ROS

The role of reactive oxygen species (ROS) in pathophysiology is receiving a lot of attention [51-55], especially the relevance of the redox state to aging [56-59]. Angiotensin II strongly stimulates the production of ROS [60]. Angiotensin II induces the transcription of several protein components of NADH oxidase and NAD(P)H oxidase present beneath the plasma membrane of endothelial cells [61,62], vascular smooth muscle cells [63,64], and adventitial fibroblasts [65]. Angiotensin II stimulates expression of the same enzymes located within the phagosomes of neutrophils [66]. Although not yet studied, the same is expected to be true for the phagosomes of macrophages.

Macrophages express sACE on their surface membrane when activated [67]. An important role of phagocytic cells such as macrophages and neutrophils is to degrade chemically supramolecular structures using ROS, e.g. O₂ and hydrogen peroxide. Targets of phagocytes include viruses, bacteria, and other large, supramolecular aggregates. The latter include lipoproteins in atheromatous plaques [68], β-amyloid peptide aggregates in Alzheimer's disease plaques [69,70], and aggregates of huntingtin in Huntington's disease [71]. As discussed above, sACE on macrophages (called “microglia” in the brain) may remain active during such an oxidant storm by having its redox-sensitive cystine “latches” buried in the interior of the autodimer, as part of the “disulfide zipper.” Unfortunately, bystander cells, especially neurons, suffer ROS-mediated apoptosis [72].

Activation of sACE by reductants, as well as by mechanical turbulence [4], could explain the enzyme's central position in pathophysiology. As mentioned above for tACE, maximal activity of sACE in situ may be realized only when both the “lid” is opened by turbulent flow and the “side wall” is opened in response to reductants. sACE has not yet been assayed in such conditions.

Nevertheless, how sACE is activated in situ may help explain such diverse phenomena as erythropoetin production by the renal “critmeter” [73], ventilation/perfusion (“V/Q”) matching in the lung [74], activation of the immune response during infection [75-78], production of myeloid cells in the bone marrow [79,80], and the vicious cycle of ischemia, thrombosis, endothelial cell apoptosis, and vascular collapse seen in conditions such as sickle cell crisis, malignant hypertension, and disseminated intravascular coagulation (DIC) [81]. In addition, sACE is an excellent candidate gene responsible for most age-related diseases as well as aging itself in all vertebrates [4,16,20,82].

Homocysteine

Normally, the extracellular milieu is oxidizing, while the intracellular milieu is reducing [51,52]. Homocysteine, which contains a free sulfhydryl group, may react extracellularly in a similar way to intracellular glutathione. Oxidation of homocysteine to the disulfide, homocystine, results in the generation of a reducing equivalent (hydride ion) which could reduce one of the “latch” cystines (C³⁵⁹—S—S—C³⁷⁷ or C³⁵⁹—S—S—C³⁷⁷), activating sACE according to the scheme in FIGS. 2, 3, 6, and 7. This could explain the association of high homocysteine levels with accelerated atherosclerosis and cancer [83,84], diseases also linked to overactivity of ACE [82].

Angiotensin II vs. NO

Angiotensin II defends the integrity of the vasculature [4] as a vasoconstrictor, pro-thrombosic agent, and vascular smooth muscle cell mitogen or angiogenesis factor [85]. Operating through its type 1 receptor, angiotensin II stimulates expression of, or sensitivity to, potent pressors such as endothelin [86], thrombin [87-89], thromboxane [90], epinephrine [91], and EPO [92]. Angiotensin II stimulates thrombosis in a number of ways: by inducing expression of the thrombin receptor and potentiating the action of thrombin [87-89], by stimulating the release of platelet activating factor (PAF) [93], and by stimulating platelet aggregation and adhesion directly [94-97]. Finally, angiotensin II stimulates the expression of the potent angiogenic factors vascular endothelial growth factor (VEGF) [98] and epiregulin [99], and acts as a potent angiogenesis factor in its own right [100], stimulating endothelial cell proliferation through activation of NF-κB [101].

Angiotensin II type 2 receptors, on the other hand, mediate apoptosis of endothelial cells [102] and other cell types such as type II pneumocytes [103, 104].

In contrast to angiotensin II, nitric oxide (NO) is the primary endothelium-derived vasodilator [105]. NO can sometimes remove cells from proliferation or apoptosis [106], cellular programs which angiotensin II initiates.

Angiotensin II and NO are biological antagonists involved in a complex balance [107, 108]. For example, angiotensin II can stimulate expression of all three isoforms of is oxide synthase to increase NO [108]. Yet ROS generated by xanthine oxidase, NAD(P)H and NADH oxidases in response to angiotensin II degrade NO and diminish NO signaling [109, 110]. This appears to be the mechanism for impaired vasodilation in patients with essential hypertension [111, 112].

Below, we explore how NO may directly inactivate sACE, the rate-limiting step for angiotensin II production by endothelial cells.

Redox Sensing by sACE and the Renal “Critmeter”

The body's degree of tissue oxygenation is sensed in the outer medulla of the kidney, where erythropoietin (EPO) is made [73]. The signal for transcription of the EPO gene appears to be angiotensin II [113-115]. Patients who lack renal function require exogenous EPO. But some patients, after receiving a kidney transplant, develop erythrocytosis with high endogenous EPO levels [116]. EPO levels and the hematocrit can be reduced with an ACE inhibitor [117], specifically, with an angiotensin II type 1 receptor antagonist [118].

The implication is that production of EPO by the renal “critmeter” is normally driven by angiotensin II through its type 1 receptor, but in some kidney transplant recipients a negative feedback loop fails to shut off angiotensin II production and therefore EPO production. We shall see in more detail below how oxygenation inactivates sACE, the rate-limiting step for production of angiotensin II.

Chronically elevated renal tissue levels of angiotensin II may be due to ongoing hypertrophy of the renal transplant [119]. The signal for renal hypertrophy appears to be production of angiotensin II by sACE in the proximal tubular brush border membrane, which can diffuse into the inner medulla [16].

Hypoxia inducible factor (HIF-Iα and related proteins) is often claimed to be the trigger for EPO production [120]. But HIF is induced several-fold more by angiotensin II than by hypoxia [121, 122]. Therefore, quantitatively speaking, HIF operates downstream from angiotensin II in the generation of EPO.

The oxygen sensor for EPO production is likely to be hemoglobin itself [123, 124]. Stamler and colleagues have shown that oxygenation of hemoglobin can displace NO from the heme ring, where it is bound in the absence of oxygen [123]. NO then becomes bound to a free cysteine sulfhydryl group on hemoglobin (“protein S nitrosylation”) [125]. NO is then transferred through a series of free cysteine sulfhydryl groups from hemoglobin in the interior of the erythrocyte to the cell exterior via the anion exchanger AE I, an abundant red cell membrane-spanning protein whose cytoplasmic tail contains numerous cysteines and binds hemoglobin [126]. From AE 1, the NO group could easily be transferred to albumin [127] (FIG. 8), and thence to a free cysteine on sACE, preventing the ability of sACE to engage in redox reactions (FIG. 9).

The possible involvement of albumin in the inactivation of sACE may explain the relative vasoconstriction and decreased effective intra-arterial volume seen in hypoalbuminemic states [128-130].

Nitrosylation of C⁴⁸⁸ on sACE may limit its ability to receive and tunnel reducing equivalents (FIG. 9). If the disulfide zipper becomes undone, S-nitrosylation of cysteines 340 or 361, or of equivalent cysteines on the C-terminal domain (FIG. 9) would inactivate sACE's “swinging gates” directly.

In the absence of sufficient oxygen, sACE escapes inactivation by NO. Instead, we hypothesize that sACE is activated by the reducing conditions of hypoxia. Assuming all reactions are at equilibrium, a four-fold decline in tissue oxygen concentration from 80 mm Hg to 20 mm Hg in the inner medulla of the kidney would be expected to increase the fraction sACE_{red}/sACEₒₓ by a factor of 4. After production by endothelial or even proximal tubular brush border membrane sACE [16], angiotensin II could easily diffuse into the renal interstitium to activate the fibroblast-like cells which make EPO [120].

Myeloid Cell Production

The ACE DID genotype is associated with chronic leukemias and lymphomas, as well as myelofibrosis and myelodysplasia [82], suggesting that angiotensin II stimulates the proliferation of bone marrow-derived cells.

This hypothesis has not yet received much study. It is known that hematopoetic cell precursors are stimulated by angiotensin II, perhaps through an oxygen-sensing, NO-mediated sACE system as described above [79,80]. Angiotensin II stimulates maturation, proliferation, and migration of dendritic cells, which originate in the bone marrow [131-133]. Angiotensin II activates NF-κB in neutrophils [66] and monocyte/macrophages [134], and so may enhance proliferation of their myeloid precursors, since NF-κB is associated with immunocyte proliferation, maturation, and activation [135-138].

Pulmonary sACE: Involved in V/Q Matching?

Activation of sACE by reducing equivalents, and inhibition of sACE by NO via oxygenated hemoglobin as discussed above, could explain how the lung matches ventilation (V) to perfusion (Q). To maximize tissue oxygenation, the lung rewards only alveoli engaged in productive gas exchange with blood flow. Vessels supplying non-functional alveoli, in contrast, undergo vasoconstriction. The mechanism for matching Q to V has not been fully described, although depolarization of vascular smooth muscle cells via inhibition of voltage-gated K⁺ channels appears to be involved [139]. Interestingly, this effect is mediated by PKC [139], so it may represent an event downstream of signaling by angiotensin II, as we shall now discuss.

sACE is present on endothelial cell membranes of pulmonary arterioles and capillaries. Pulmonary vessels are located in the interstitium, not more than a few cell widths away from the gaseous phase in neighboring alveoli. Oxygen diffuses from the alveolus to nearby blood vessels, is picked up by hemoglobin, and is pumped by the left ventricle to the rest of the body.

If the pulmonary capillary has no oxygen to pick up because the alveolus nearby is non-functional, then the oxygen tension in the interstitium surrounding that alveolus will fall, the carbon dioxide tension rise, and the pH will fall. Both lower oxygen tension and higher CO₂ tension constitute reducing conditions, which, we postulate, should reduce the cystine bridges in sACE to free sulfhydryl groups. The side wall for each active site will fall apart, exposing the active site (FIGS. 2,3,6,7).

Local angiotensin II production should increase, causing the vessel to constrict. In part, this may be mediated by depolarization of smooth muscle cells in response to PKC-mediated inhibition of ATP-sensitive, voltage-gated K⁺ channels [139]. Turbulent blood flow may further activate sACE by causing the enzyme's “lids” to open.

When gas exchange improves, oxygen tension in the interstitium and at the plasma membrane of the endothelial cell should increase, the free cysteine sulfhydryl groups of sACE should become oxidized to cystine once again, and the side walls of sACE should get “locked” up and inactivated. Nitric oxide (NO) may also contribute significantly to the inactivation of sACE, as discussed above (FIGS. 8,9).

As a result, angiotensin II production should drop in a pulmonary capillary next to a functioning alveolus. The balance between vasoconstriction, mediated by angiotensin II and “downstream” vasoconstrictors whose expression is induced by angiotensin II, such as endothelin [86], and vasodilation, mediated by NO, should shift in favor of vasodilation. Furthermore, the gain in the system will be multiplicative [34]. The pulmonary capillary will dilate, and blood flow will again resume to the functional alveolus.

If alveolar gas exchange is impaired for a long time (days), long-term effects of angiotensin II operating through AT 1 receptors include hyperplasia of vascular smooth muscle cells leading to pulmonary hypertension, and elaboration of TGF-β proliferation of interstitial fibroblasts, leading to pulmonary fibrosis [140]. Angiotensin II-mediated induction of sACE will amplify this positive feedback loop [141].

Over days, angiotensin II may stimulate apoptosis of alveolar epithelial cells [103,104] and loss of pulmonary parenchyma, the hallmark of emphysema [142]. Under the constant driving pressure of angiotensin II, some alveolar epithelial cells may escape from growth control (apoptosis) and become cancerous [82,143].

Because of sACE's key role in V/Q matching, effective inhibition of tissue ACE or antagonism of angiotensin II type 1 receptors by ARBs is expected to be useful for any pulmonary disease in which gas exchange is impaired. In addition to promoting vasoconstriction and pulmonary hypertension, as well as alveolar epithelial apoptosis, angiotensin II also appears to be a major cytokine (see below). Examples of diseases which are likely to benefit from angiotensin II blockade include emphysema [20], bronchiolitis obliterans especially after respiratory syncytial virus [144], cystic fibrosis [145], acute respiratory distress syndrome and smoke inhalation [146], severe acute respiratory syndrome (SARS) [147], radiation pneumonitis [148], and other forms of interstitial lung disease with pulmonary fibrosis. Lung cancers which may initially arise due to hypoxemia-induced production of angiotensin II might be delayed or perhaps prevented altogether with an ACE inhibitor or ARB [82].

The molecular mechanism described above fails to explain how hyperoxia, as in prolonged mechanical ventilation using an F_{I}O2≧0.5, could result in pulmonary fibrosis. Indeed, both active sites of ACE should be “locked up” by hyperoxia-mediated oxidation of key cystines. Recent evidence suggests that hyperoxia mimics the action of angiotensin II by activating AP-1 directly [149], although the mechanism is unclear.

sACE in Pathology

1. Diabetes

Diabetes, hypertension, and their complications could all result, at least in part, from activation of sACE [4,16,20,82]. In diabetes, sACE is activated by more than increased plasma osmolality, since hyperglycemia contributes relatively little (<5%) to plasma osmolality [4]. But sugars are potent reducing agents [150], and there is considerable evidence linking glucose concentration to the risk of developing diabetic complications [151].

According to the hypothesis presented here, tripling of the serum glucose concentration from 100 mg/dl to 300 mg/dl would result in tripling of the ratio of sACE_{red}/sACEₒₓ, where sACE_{red} represents the reduced and fully activated form of sACE. If sACE normally exists in the ratio of 1:9, e.g. 10% reduced (constitutively active) and 90% oxidized (activated only by mechanical flow), then tripling the glucose concentration will change the ratio to 1:3, i.e. 25% reduced (constitutively active) and 75% oxidized (activated only by mechanical flow). The effect of hyperglycemia will thus be to increase the fraction of reduced, fully activated sACE from 10% to 25%, a 2.5-fold change. In the limit, the change in sACEred will be the same as the change in glucose concentration; e.g., for 1% sACE_{red}, or 1:99, tripling the glucose concentration will triple the fraction of sACE_{red} to 1:33, or 3%.

Generation of angiotensin II could explain downstream events observed in diabetes, such as activation of protein kinase C and TGF-β [152, 153]. In type II diabetes mellitus, angiotensin II appears to be involved in a positive feedback loop. Angiotensin II, operating through PKC, inhibits signaling by the insulin receptor by phosphorylation of one or more key serine residues [154]. Reduced responsiveness to insulin causes serum glucose levels to rise, which may further activate sACE through the redox mechanism postulated above. Angiotensin II levels rise, further activating protein kinase C and interfering with insulin sensitivity, establishing a vicious cycle.

Experimentally, ACE gene expression and activity is increased after the initiation of streptozotocin-induced diabetes [155, 156]. As mentioned above, angiotensin II induces expression of sACE via the ATI receptor and PKC [157], contributing to this positive feedback loop.

This may explain the “metabolic syndrome” (also called “syndrome X”), i.e. essential hypertension and insulin resistance [158]. As tissue angiotensin II levels rise, so will insulin resistance. The amplitude of the excursions in the plasma insulin concentration will therefore increase accordingly. Once β-islet cells begin undergoing apoptosis due to severe insulin overshoot and hypoglycemia, the metabolic syndrome is well on its way to becoming clinically overt type II diabetes mellitus [16].

2. Gout

Hyperuricemia and gout are features of the metabolic syndrome, and are associated with cardiovascular disease and type II diabetes [159]. These diseases are all associated with the ACE D/D genotype [82], a marker of excessive tissue ACE activity.

Uric acid is produced from xanthine and hypoxanthine by the enzyme xanthine oxidase (XO). Angiotensin II stimulates XO production by endothelial cells [160], perhaps in an autocrine/paracrine fashion. XO is expressed on the plasma membrane of endothelial cells in the same location as ACE. Uric acid can function as an anti-oxidant (reducing agent), and appears to activate ACE directly [161], creating the possibility of a vicious cycle: angiotensin II →↑XO→↑uric acid→↑sACE→↑angiotensin II.

XO can create uric acid through electron transfer to its molybdenum(VI) center, thence to an iron-sulfur protein, and thence to a flavin moiety. But XO can also create free oxygen radicals through its flavin center alone. These free oxygen radicals deplete NO by creating peroxynitrite (ONOO—) [109,110,159,161]. Thus, synthesis of XO appears to be yet another mechanism for the vasoconstrictor, prothrombotic, profibrotic, proapoptotic pathway initiated by angiotensin II to the battle against the vasodilatory, antithrombotic, antiproliferative and antifibrotic pathway controlled by NO.

3. Vicious Cycles Leading to Vascular Collapse: Sepsis DIC Malignant Hypertension Sickle Cell Disease Pre-Eclampsia

Reducing conditions (low oxygen tension, low pH) exist commonly in tissue vascular beds, such as liver and muscle, during conditions of hypoperfasion, e.g. in cardiogenic, hypovolemic, or septic shock; sickle cell disease; malignant hypertension; disseminated intravascular coagulation (DIC); and pre-eclampsia. In these diseases, sACE should be maximally activated by redox conditions and mechanical turbulence.

DIC often leads rapidly to death in patients with sepsis, shock, or malignant hypertension. The essence of DIC, hypercoagulation, could arise by a profound imbalance between angiotensin II, which is pro-thrombotic [162], and NO, which is antithrombotic [163]. In addition, endothelial cell ischemia and apoptosis expose prothrombotic tissue factors [164] and promote coagulation on the vascular wall.

Sickle cell “crisis” is similar to DIC since it involves a vicious cycle of vasoconstriction, hypoperfusion, local hypoxia and acidemia, and further sickling of red cells. Stiff, non deformable sickled red cells scrape against the vascular wall [165,166], reducing the unstirred layer from 1 μm to perhaps 10 nm, the approximate dimension of the sACE molecule protruding from the endothelial cell plasma membrane [4].

In patients with sickle cell disease, sACE molecules on the surface of the endothelial cell are exposed to more shear stress than usual [165, 166]. With a reduction in the thickness of the unstirred layer, sACE molecules in vessels usually exposed to laminar blood flow may be exposed to shear stress. If sACE is activated by mechanical flow, then endothelial sACE molecules in much of the vasculature will be activated. Increased local production of angiotensin II will result, leading to vasoconstriction, hypoxia, acidemia, and further sickling of erythrocytes.

Sickled cells release extracellular hemoglobin, which traps NO [167]. NO-mediated inactivation of sACE should decrease, with the angiotensin II-NO balance tilting further towards angiotensin II, promoting the vicious cycle.

Effective tissue ACE inhibition [20] or blockade of angiotensin II type 1 receptors, either orally or intravenously (the latter for patients who are vomiting and cannot keep pills down) is therefore proposed as prophylaxis against sickle cell crisis, as well as a treatment for it.

In malignant hypertension, shear stress is increased because of the abnormally high systemic blood pressure, not because of the scrubbing action of sickled erythrocytes. With higher blood velocity and shear stress, the unstirred layer is also reduced, leading to the same picture as sickle cell crisis described above. ACE inhibition or angiotensin II blockade should therefore be of special effectiveness in the clinical management of malignant hypertension, and DIC.

Pre-eclampsia is another vaso-occlusive disease which appears to result from a vicious cycle favoring the production of angiotensin II over NO [168, 169]. ACE inhibition or angiotensin II blockade should therefore also be effective in the clinical management of pre-eclampsia.

4. Role of sACE in Inflammation and Autoimmunity

Angiotensin II already functions as a cytokine in invertebrates [170]. In vertebrates, it is a pyrogen [171]. sACE appears, as CD 143, on the plasma membrane of activated macrophages [172] and T lymphocytes [173]. T cells can stimulate the expression of sACE on monocytes in an MHC-restricted manner [174]. This apparently involves cell-cell contact (FIG. 10) and induction of sACE via the AT1 receptor [157] and PKC [175].

Angiotensin II is a potent cytokine [176], capable of stimulating the synthesis of macrophage migration inhibition factor (MIF) [177], TNF-α [178], MCP-1 and TGF-β [179], among other cytokines.

Bacterial infection lowers tissue oxygen tension. Bacteria either consume oxygen themselves, or require an anerobic environment to replicate. Along with tissue hypoxia, bacteria produce lactic acid, lowering tissue pH. (An exception are the urease-producing bacteria in the urinary tract). Under such reducing conditions, sACE on the surface membrane of macrophages or T cells should become activated according to the hypothesis presented here.

Angiotensin II stimulates the production of the antiviral protein interferon-y from T cells [180], which interferes with viral replication. Angiotensin II helps promote apoptosis [181], especially of virally infected cells [182], further limiting viral replication.

In addition, sACE may permit tight cellular interactions. Binding of the N-terminal domain of sACE on one cell (macrophage or T cell) with the C-terminal domain of sACE on another cell (macrophage, T cell, or endothelial cell, for example) may promote specific cell-cell binding (FIG. 11).

Overactivity of ACE has been associated with autoimmune diseases such as rheumatoid arthritis [82, 183], lupus [82, 184], and fibromyalgia/chronic fatigue syndrome [185]. We have observed gratifying clinical responses to angiotensin II antagonism in patients with T-cell disorders such as psoriasis (FIG. 12) and alopecia areata, as well as viral diseases characterized by an overly exuberant host response such as West Nile virus encephalitis (Table 3). A similar approach may work for SARS [147].

HIV infectivity and progression to AIDS are also associated with the ACE D/D genotype [82]. This is perhaps not surprising considering that retroviruses require proliferating cells for their replication [186], and angiotensin II stimulates proliferation of macrophages [138, 176, 187] where HIV replicates for its first several months in a human host [188], as well as T cells [173], HIV's eventual home [188].

Angiotensin II blockade may also be of benefit in the eradication of Mycobacterium species. Like HIV [82], M tuberculosis [82] and M leprae proliferate within activated monocytes and dendritic cells. These cells are activated by angiotensin II [131, 172].

Finally, angiotensin II blockade may be beneficial against hepatitis A and B [82]. Hepatic stellate cells have been implicated in hepatitis [189], and are specifically activated by angiotensin II [190-192]. The same approach may help in pancreatitis [193], especially for women [82].

5. Role of sACE in Cancer

Overactivity of sACE is associated with all solid and hematogenous cancers except prostate cancer, in which sACE activity is actually protective [82]. Vascular signaling by sACE thus appears to drive most cancers. For example, many solid cancers, including colon, have recently been shown to be initiated by Wnt, which acts upstream of beta-catenin and APC [194]. Wnt is activated by PKC [195], so that angiotensin II production by vascular sACE operates upstream of Wnt.

Breast cancer has recently been associated with the ACE D/D genotype In Chinese women [196]. Since angiotensin II promotes cell proliferation and angiogenesis, ACE inhibitors or ARBs may be useful adjunctive treatment for these cancers [197, 198].

Here we report a single case of a 67 year old white woman with unresectable pancreatic cancer who has been treated with high dose 5-fluorouracil (5-FU), 500 mg/m² and Naitrexone 5 mg at bedtime since partial resection of her tumor in February, 2002. In November, 2002, she began taking quinapril for hypertension; she currently takes 60 mg with good blood pressure control. She is still alive in November, 2003, and her appetite remains excellent. The median survival of similar patients is less than 4 months, and cachexia is common [199,200]. Presumably, quinapril inhibited TNF-a production by her monocytes [178].

Prostate cancer in white men is a notable exception. In black men, ACE overactivity is associated with prostate cancer and PSA level, as in other cancers [82, 196]. In white men, however, the ACE D/D genotype is associated with benign prostatic hyperplasia, but negatively associated with prostate cancer and PSA level [82], suggesting that angiotensin II promotes hyperplasia but guards against neoplasia of epithelial cells in white men.

sACE is highly expressed in the glandular epithelium of benign prostatic hyperplasia [201]. Patients at a predominantly white hospital who take an ACE inhibitor are at significantly higher risk of also having prostate cancer (Table 3). The implication is that white men taking an ACE inhibitor should be followed closely with a PSA test.

The explanation for the unexpected protection against prostate cancer and the negative association of the ACE D/D genotype with PSA level in white men may lie with Nkx3.1, a prostate-specific inhibitor of prostate cancer [202]. The Nk×3.1 promoter (GenBank NM 006167) has a single TPA response element (TRE, aatetacaatgattcaaaaga) located 1.6 kb 5′ to the translation start site [203]. This THE could be activated by AP-1, acting downstream of angiotensin II, the angiotensin II type 1 receptor [204], and PKC. There are two additional TRE's located at −5.7 kb and −9 kb upstream, with the −9 kb site having two overlapping TRE's. However, these additional TRE's are likely to be too far removed from the transcription start site to influence gene expression.

A possible genetic pathway for the initiation of BPH and prostate cancer in white men is presented in FIG. 12. Testosterone stimulates the production of both tACE [206] and sACE [207], renin [208], Nkx3.1 [209], the prostate tumor suppressor gene [210], and prostate-derived ets factor (PDEF) which promotes prostate cancer [211]. PDEF drives PSA production [212] as well as progression to cancer. PDEF expression is inhibited by Nkx3.1 [212]. Angiotensin II stimulates the expression of Nkx3.1 via PKC (discussed above), and inhibits the action of PDEF [213], which could explain the protective role of the ACE D/D genotype In prostate cancer. Angiotensin II inhibits expression of PSA via the AT1 receptor [213]. Finally, estrogen inhibits the expression of sACE [214], which might be expected to limit its efficacy in prostate cancer (FIG. 11, [215]).

6. Neurodegenerative Diseases

As mentioned above, neurons are sensitive to apoptotic signals such as intracellular redox imbalance and ROS [216]. It is unclear whether they are especially sensitive, or whether their cellular metabolism is higher than most other cell types, leaving little room for additional insults.

Neuronal apoptotis in response to ROS is thought to contribute to all neurodegenerative diseases, including Alzheimer's disease, Parkinson's disease, Huntington's disease, amyotrophic lateral sclerosis (ALS) [217,218], the retinal degeneration seen in age-related macular degeneration and retinitis pigmentosa [219], and diabetic neuropathy [220-223]. Diabetic neuropathy has already shown improvement with ACE inhibitors [224,225], as expected [226].

7. sACE and Aging

Besides being associated with most common diseases of aging [82], overactivity of sACE is consistent with most current theories of aging [227,228,229].

For example, calorie restriction prolongs life-span in a number of species [230]. With less fuel consumption, mitochondrial electron transport and production of ROS are decreased [231]. sACE overactivity as a cause of aging is entirely consistent with this model, since angiotensin II stimulates mitochondria, electron transport, oxygen consumption [232], and production of ROS [233,234]. Chronic angiotensin II signaling leads to mitochondrial hypertrophy and proliferation [235]. Eventually, angiotensin II leads to mitochondrial dysfunction, with increased uncoupling of electron transport from ATP synthesis, and increased production of ROS. Inhibition of ACE in old animals restores mitochondrial function [236].

Mutations in the insulin-like receptor of Drosophila and daf-2 in C. elegans are associated with extended lifespan but small size [229,237]. As in rodents, growth and metabolism limit lifespan. Because they result in decreased fuel consumption, these mutations are equivalent to calorie restriction in rodents.

Werner syndrome, characterized by accelerated aging, is due to a mutation in a DNA helicase. DNA helicases like wrn are the cell's first defense against DNA damage induced by ROS [238], confirming that senescence is promoted by ROS.

In vitro, telomerase activity is required for replicative competence of cells in culture [239]. Angiotensin II can repress telomerase activity indirectly through TGF-β and p53 [239,240], among other pro-apoptotic factors.

Osteoarthritis [82], skeletal muscle wasting and cachexia are features of old age. Skeletal muscle wasting can be due to angiotensin II, which opposes the action of IGF-1 [241]. Cachexia can result from a high circulating level of TNF-α [242], derived from monocytelmacrophages stimulated by angiotensin II [243].

In summary, population morbidity and mortality should be significantly reduced, and longevity enhanced, by widespread use of an ACE inhibitor or ARB. The only caveat is that white men taking an ACE inhibitor or ARB will need to check their PSA at least once a year.

TABLE 1
ACE inhibitors, listed in decreasing order of hydrophobicity at
pH 7.4. The active moiety, not the pro-drug, is considered. Data
are calculated from [23] or [24]. Discrepancies, which
are occasionally marked, reflect different assay conditions.

| | P(octanol:water) | | | |
|---|---|---|---|---|
| Trandolaprilat | 10.47 | [23] | | |
| Quinaprilat | 6.6 | [23] | | |
| Benazeprilat | 3.0 | [23] | | |
| Fosinoprilat | — | | 0.33 | [24] |
| Zofenoprilat | — | | 0.22 | [24] |
| Ramiprilat | 2.6 | [23]; | 0.011 | [24] |
| Cilazaprilat | 1.7 | [23] | | |
| Enalaprilat | 0.62 | [23]; | <0.001 | [24] |
| Perindoprilat | 0.44 | [23] | | |
| Lisinopril | 0.05 | [23]; | <0.001 | [24] |
| Captopril | 0.01 | [23]; | 0.004 | [24] |
| Ceronapril | — | | <0.001 | [24] |

TABLE 3
Pharmacy data from a predominantly white midwestern veterans′ hospital
population. Goserelin acetate, an LH-RH agonist, was chosen as a marker
drug for prostate cancer, since this is the only indication for the
drug's use [203]. ACEI, ACE inhibitor.

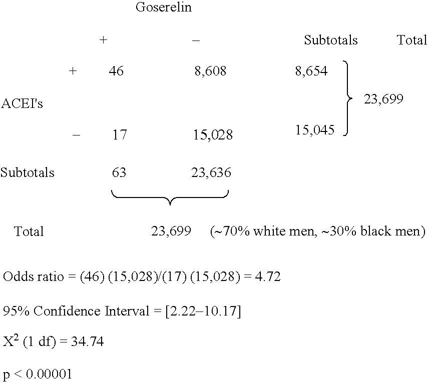

### REFERENCES

1. Sturrock, E. D.; Yu, X. C.; Wu, Z.; Biemann, K.; Riordan, J. F. Assignment of Free and Disulfide-Bonded Cysteine Residues in Testis Angiotensin-Converting Enzyme: Functional Implications. Biochemistry, 1996, 35, 9560-9566.
2. Natesh, R.; Schwager, S. L.; Sturrock, E. D.; Acharya, K. R. Crystal Structure of the Human Angiotensin-Converting Enzyme-Lisinopril Complex. Nat., 2003, 421, 551-4.
3. Hooper, N. M.; Turner, A. J. An ACE Structure. Nat Struct Biol., 2003, 10, 155-157.
4. Moskowitz, D. W. Is “Somatic” Angiotensin I-Converting Enzyme a Mechanosensor? Diabetes Technology & Therapeutics 2002, 4, 841-858.
5. Guex, N. and Peitsch, M. C. SWISS-MODEL and the Swiss-PdbViewer: An environment for comparative protein modeling. Electrophoresis, 1997, 18, 2714-2723. http://www.expasv.org/spdbv/
6. Michel, B; Nirina, L. B.; Grima, M.; Ingert, C.; Coquard, C.; Barthelmebs, M.; Imbs, J. L. [Effects of hydroxyl radicals on purified angiotensin I converting enzyme] Arch Mal Coeur Vaiss., 1998, 91,1013-1019.
7. Yang, K. S.; Kang, S. W.; Woo, H. A.; Hwang, S. C.; Chae, H. Z.; Kim, K.; Rhee, S. G. Inactivation of Human Peroxiredoxin I during Catalysis as the Result of the Oxidation of the Catalytic Site Cysteine to Cysteine-Sulfinic Acid. J Biol Chem., 2002, 277, 38029-38036.
8. Pauls, K.; Metzger, R.; Steger, K.; Klonisch, T.; Danilov, S.; Franke, F. E. Isoforms of Angiotensin I-Converting Enzyme in the Development and Differentiation of Human Testis and Epididymis. Andrologia, 2003, 35, 32-43.
9. Rigau, T.; Rivera, M.; Palomo, M. J.; Fernandez-Novell, J. M.; Mogas, T.; Ballester, J.; Pena, A.; Otaegui, P. J.; Guinovart, J. J.; Rodriguez-Gil, J. E. Differential effects of glucose and fructose on hexose metabolism in dog spermatozoa. Reproduction, 2002, 123, 579-591.
10. Vinson, G. P.; Puddefoot, J. R.; Ho, M. M.; Barker, S.; Mehta, J.; Saridogan, E.; Djahanbakhch, O. Type 1 Angiotensin II Receptors in Rat and Human Sperm. J Endocrinol., 1995, 144, 369-378.
11. Hamasaki, M.; Wakimoto, M.; Maehara, T.; Matsuo, H. Three-dimensional structures of the neck region of the hamster spermatozoa in the caudal epididymis. Arch Histol Cytol., 1994, 57, 59-65.
12. Kaneko, S.; Oshio, S.; Kobayashi, T.; Iizuka, R. Effects of angiotensins on the motility of human sperm. J Pharmacobiodyn., 1984, 7,87-93.
13. Gaddum-Rosse, P.; Blandau, R. J.; Lee, W. I. Sperm Penetration into Cervical Mucus in vitro. I. Comparative Studies. Fertil Steril, 1980, 33, 636-643.
14. Heikinheimo, O.; Gibbons, W. E. The Molecular Mechanisms of Oocyte Maturation and Early Embryonic Development are Unveiling New Insights into Reproductive Medicine. Mol Hum Reprod., 1998, 4, 745-756.
15. Li, X. F.; Ahmed, A. Dual Role of Angiotensin II in the Human Endometrium. Hum Reprod., 1996, 11 Suppl 2, 95-108.
16. Moskowitz, D. W. Pathophysiologic Implications of Angiotensin I-Converting Enzyme as a Mechanosensor: Diabetes. Diabetes Technology & Therapeutics 2003, 5, 189-199.
17. Velletri, P. A.; Billingsley, M. L.; Lovenberg, W. Thermal denaturation of rat pulmonary and testicular angiotensin-converting enzyme isozymes. Effects of chelators and CoC 12. Biochim Biophys Acta., 1985, 839, 71-82.
18. Voronov, S.; Zueva, N.; Orlov, V.; Arutyunyan, A.; Kost, O. Temperature-induced selective death of the C-domain within angiotensin-converting enzyme molecule. FEBS Lett., 2002, 522, 77-82.
19. Binevski, P. V.; Sizova, E. A.; Pozdnev, V. F.; Kost, O. A. Evidence for the Negative Cooperativity of the Two Active Sites within Bovine Somatic Angiotensin-Converting Enzyme. FEBS Lett., 2003, 550, 84-88.
20. Moskowitz, D. W. From pharmacogenomics to improved patient outcomes: angiotensin 1-converting enzyme as an example. Diabetes Technol Ther., 2002, 4, 519-532.
21. Houben, A. J.; Kroon, A. A.; de Haan, C. H.; Fuss-Lejeune, M. J.; de Leeuw, P. W. Quinaprilat-induced vasodilatation in forearm vasculature of patients with essential hypertension: comparison with enalaprilat. Cardiovasc Drugs Ther, 2000, 14, 657-663.
22. Guazzi, M.; Pontone, G.; Trevisi, N.; Lomanto, M.; Meizi, G.; Agostoni, P. [Effectiveness of long-term ACE-inhibition on pulmonary diffusion and ventilation-perfusion ratio in chronic heart failure: correlation with physical performance] Ann Ital Med Int, 1998, 13, 17-23.
23. Genes, N.; Lemoine, G.; Duguet, A.; Vaur, L.; Moreau, G.; Etrillard, C.; Souiller, F. Comparative lipophilicity of trandolapril and other conversion enzyme inhibitors. Thérapie, 1995, 50, 131-136.
24. Ranadive, S. A.; Chen, A X; Serajuddin, A. T. Relative Lipophilicities and Structural-Pharmacological Considerations of Various Angiotensin-Converting Enzyme (ACE) Inhibitors. Pharm Res., 1992, 9, 1480-1486.
25. Dzau, V. J. Circulating versus local renin-angiotensin system in cardiovascular homeostasis. Circulation., 1988, 77, I 4-I 13.
26. Dzau, V. J.; Bernstein, K.; Celermajer, D.; Cohen, J.; Dahlof, B.; Deanfield, J.; Diez, J.; Drexler, H.; Ferrari, R.; Van Gilst, W.; Hansson, L.; Hornig, B.; Husain, A.; Johnston, C.; Lazar, H.; Lonn, E.; Luscher, T.; Mancini, J.; Mimran, A.; Pepine, C.; Rabelink, T.; Remme, W.; Ruilope, L.; Ruzicka, M.; Schunkert, H.; Swedberg, K.; Unger, T.; Vaughan, D.; Weber, M. Pathophysiologic and therapeutic importance of tissue ACE: a consensus report. Cardiovasc Drugs Ther., 2002, 16, 149-160.
27. Johnston, C. I.; Fabris, B.; Yamada, H.; Mendelsohn, F. A.; Cubela, R.; Sivell, D.; Jackson, B. Comparative studies of tissue inhibition by angiotensin converting enzyme inhibitors. J Hypertens Suppl., 1989, 7, S11-S16. Erratum in: J Hypertens Suppl, 1991, 9, following H30.
28. Kaplan, H. R.; Taylor, D. G.; Olson, S. C.; Andrews, L. K. Quinapril—a preclinical review of the pharmacology, pharmacokinetics, and toxicology. Angiology, 1989, 40, 335-350.
29. Fabris, B.; Chen, B. Z.; Pupic, V.; Perich, R.; Johnston, C. I. Inhibition of angiotensin-converting enzyme (ACE) in plasma and tissue. J Cardiovasc Pharmacol., 1990, 15 Suppl 2, S6-S13.
30. Nakajima, T.; Yamada, T.; Setoguchi, M. Prolonged inhibition of local angiotensin-converting enzyme after single or repeated treatment with quinapril in spontaneously hypertensive rats. J Cardiovasc Pharmacol., 1992, 19, 102-107.
31. Deddish, P. A.; Wang, L X; Jackman, H. L.; Michel, B.; Wang, J.; Skidgel, R. A.; Erdos, E. G. Single-domain angiotensin I converting enzyme (kininase II): characterization and properties. J Pharmacol Exp Ther., 1996, 279, 1582-1589.
32. Roy, A.; Solodovnikova, N.; Nicholson, T.; Antholine, W.; Walden, W. E. A Novel Eukaryotic Factor for Cytosolic Fe-S Cluster Assembly. EMBO J, 2003, 22, 4826-4835.
33. Page, C. C.; Moser, C. C.; Chen, X.; Dutton, L. Natural Engineering Principles of Electron Tunnelling in Biological Oxidation-Reduction. Nat., 1999, 402, 47-52.
34. Newsholme, E. A.; Crabtree, B. Substrate cycles in metabolic regulation and in heat generation. Biochem Soc Symp., 1976, 41, 61-109.
35. Larsen, E. C.; DiGennaro, J. A.; Saito, N.; Mehta, S.; Loegering, D. J.; Mazurkiewicz, J. E.; Lennartz, M. R. Differential requirement for classic and novel PKC isoforms in respiratory burst and phagocytosis in RAW 264.7 cells. J. Immunol., 2000, 165, 2809-2817.
36. Ehlers, M. R.; Chen, Y. N.; Riordan, J. F. Spontaneous solubilization of membrane-bound human testis angiotensin-converting enzyme expressed in Chinese hamster ovary cells. Proc Natl Acad Sci USA, 1991, 88, 1009-1013.
37. Oppong, S. Y.; Hooper, N. M. Characterization of a secretase activity which releases angiotensin-converting enzyme from the membrane. Biochem J, 1993, 292, 597-603.
38. Ramchandran, R.; Sen, G. C.; Misono, K.; Sen, I. Regulated cleavage-secretion of the membrane-bound angiotensin-converting enzyme. J Biol Chem, 1994, 269, 2125-2130.
39. Schwager, S. L.; Chubb, A. J.; Scholle, R. R.; Brandt, W. F.; Eckerskorn, C.; Sturrock, E. D.; Ehlers, M. R. Phorbol ester-induced juxtamembrane cleavage of angiotensin-converting enzyme is not inhibited by a stalk containing intrachain disulfides. Biochemistry, 1998, 37,15449-15456.
40. Ehlers, M. R.; Scholle, R. R.; Riordan, J. F. Proteolytic release of human angiotensin-converting enzyme expressed in Chinese hamster ovary cells is enhanced by phorbol ester. Biochem Biophys Res Commun., 1995, 206, 541-547.
41. Eyries, M.; Michaud, A.; Deinum, J.; Agrapart, M.; Chomilier, J.; Kramers, C.; Soubrier, F. Increased shedding of angiotensin-converting enzyme by a mutation identified in the stalk region. J Biol Chem, 2001, 276, 5525-5532.
42. Cole, J.; Quach, D. L.; Sundaram, K.; Corvol, P.; Capecchi, M. R.; Bernstein, K. E. Mice lacking endothelial angiotensin-converting enzyme have a normal blood pressure. Circ Res, 2002, 90, 87-92.
43. Cole, J. M.; Khokhlova, N.; Sutliff, R. L.; Adams, J. W.; Disher, K. M.; Zhao, H.; Capecchi, M. R.; Corvol, P.; Bernstein, K. E. Mice lacking endothelial ACE: normal blood pressure with elevated angiotensin II. Hypertension, 2003, 41, 313-321.
44. Lanzillo, J. J.; Stevens, J.; Dasarathy, Y.; Yotsumoto, H.; Fanburg, B. L. Angiotensin-converting enzyme from human tissues. Physicochemical, catalytic, and immunological properties. J Biol Chem, 1985, 260, 14938-14944.
45. Schullek, J. R.; Wilson, I. B. Purification of bovine angiotensin converting enzyme. Life Sci, 1989, 45, 685-90.
46. Wei, L.; Clauser, E.; Alhenc-Gelas, F.; Corvol, P. The two homologous domains of human angiotensin I-converting enzyme interact differently with competitive inhibitors. J Biol Chem, 1992, 267,13398-13405.
47. Bevilacqua, M.; Vago, T.; Rogolino, A.; Conci, F.; Santoli, E.; Norbiato, G. Affinity of angiotensin I-converting enzyme (ACE) inhibitors for N- and C-binding sites of human ACE is different in heart, lung, arteries, and veins. J Cardiovasc Pharmacol, 1996, 28, 494-499.
48. Balyasnikova, I. V.; Karran, E. H.; Albrecht, R. F. 2^{nd}; Danilov, S. M. Epitope-specific antibody-induced cleavage of angiotensin-converting enzyme from the cell surface. Biochem J, 2002, 362, 585-595.
49. Kost, O. A.; Balyasnikova, I. V.; Chemodanova, E. E.; Nikolskaya, I. I.; Albrecht, R. F. 2^{nd}; Danilov, S. M. Epitope-dependent blocking of the angiotensin-converting enzyme dimerization by monoclonal antibodies to the N-terminal domain of ACE: possible link of ACE dimerization and shedding from the cell surface. Biochemistry, 2003, 42, 6965-6976.
50. Danilov, S.; Jaspard, E.; Churakova, T.; Towbin, H.; Savoie, F.; Wei, L.; Alhenc-Gelas, F. Structure-function analysis of angiotensin I-converting enzyme using monoclonal antibodies. Selective inhibition of the amino-terminal active site. J Biol Chem, 1994, 269, 26806-26814.
51. Droge W. Free radicals in the physiological control of cell function. Physiol Rev, 2002, 82, 47-95.
52. Mikkelsen, R. B.; Wardman, P. Biological chemistry of reactive oxygen and nitrogen and radiation-induced signal transduction mechanisms. Oncogene, 2003, 22, 5734-5754.
53. Schmitz, M. L.; Bacher, S.; Droge, W. Molecular analysis of mitogen-activated protein kinase signaling pathways induced by reactive oxygen intermediates. Methods Enzymol, 2002, 352, 53-61.
54. Hildebrandt, W.; Alexander, S.; Bartsch, P.; Droge, W. Effect of N-acetyl-cysteine on the hypoxic ventilatory response and erythropoietin production: linkage between plasma thiol redox state and O(2) chemosensitivity. Blood, 2002, 99, 1552-1555.
55. Hotz-Wagenblatt, A.; Droge, W. Redox-mediated functional and structural changes in insulin receptor kinase. Methods Enzymol, 2002, 348, 288-296.
56. Droge, W. Aging-related changes in the thiol/disulfide redox state: implications for the use of thiol antioxidants. Exp Gerontol, 2002, 37, 1333-1345.
57. Droge, W. The plasma redox state and ageing. Ageing Res Rev, 2002, 1, 257-278.
58. Hildebrandt, W.; Kinscherf, R.; Hauer, K.; Holm, E.; Droge, W. Plasma cystine concentration and redox state in aging and physical exercise. Mech Ageing Dev, 2002, 123, 1269-1281.
59. Hauer, K.; Hildebrandt, W.; Sehl, Y.; Edler, L.; Oster, P.; Droge, W. Improvement in muscular performance and decrease in tumor necrosis factor level in old age after antioxidant treatment. J Mol Med, 2003, 81, 118-125.
60. Wolf, G. Free Radical Production and Angiotensin. Curr Hypertens Rep., 2000, 2, 167-173.
61. Munzel, T.; Hink, U.; Heitzer, T.; Meinertz, T. Role for NADPH/NADH Oxidase in the Modulation of Vascular Tone. Ann N YAcad Sci., 1999, 874, 386-400.
62. Lang, D.; Mosfer, S. I.; Shakesby, A.; Donaldson, F.; Lewis, M. J. Coronary Microvascular Endothelial Cell Redox State in Left Ventricular Hypertrophy: the Role of Angiotensin II. Circ Res., 2000, 86, 463-9.
63. Sorescu, D.; Somers, M. J.; Lassegue, B.; Grant, S.; Harrison, D. G.; Griendling, K. K. Electron Spin Resonance Characterization of the NAD(P)H Oxidase in Vascular Smooth Muscle Cells. Free Radic Biol Med., 2001, 30, 603-612. Erratum in: Free Radic Biol Med., 2001, 30, 1188.
64. Lassegue, B.; Sorescu, D.; Szocs, K.; Yin, Q.; Akers, M.; Zhang, Y.; Grant, S. L.; Lambeth, J. D.; Griendling, K. K. Novel gp91(phox) Homologues in Vascular Smooth Muscle Cells: noxI Mediates Angiotensin II-Induced Superoxide Formation and Redox-Sensitive Signaling Pathways. Circ Res., 2001, 88, 888894.
65. Pagano, P. J.; Chanock, S. J.; Siwik, D. A.; Colucci, W. S.; Clark, J. K. Angiotensin II Induces p67phox mRNA Expression and NADPH Oxidase Superoxide Generation in Rabbit Aortic Adventitial Fibroblasts. Hypertension, 1998, 32, 331-337.
66. El Bekay, R.; Alvarez, M.; Monteseirin, J.; Alba, G.; Chacon, P.; Vega, A.; Martin-Nieto, J.; Jimenez, J.; Pintado, E.; Bedoya, F. J.; Sobrino, F. Oxidative Stress is a Critical Mediator of the Angiotensin II Signal in Human Neutrophils: Involvement of Mitogen-Activated Protein Kinase, Calcineurin, and the Transcription Factor NF-kappaB. Blood, 2003, 102, 662-671.
67. Romer, F. K. Clinical and Biochemical Aspects of Sarcoidosis. With Special Reference to Angiotensin-Converting Enzyme (ACE). Acta Med Scand Suppl. 1984, 690, 3-96.
68. Keidar, S.; Heinrich, R.; Kaplan, M.; Hayek, T.; Aviram, M. Angiotensin II administration to atherosclerotic mice increases macrophage uptake of oxidized LDL: a possible role for interleukin-6. Arterioscler Thromb Vasc Biol, 2001, 21, 1464-1469.
69. Johnstone, M.; Gearing, A. J.; Miller, K. M. A central role for astrocytes in the inflammatory response to beta-amyloid; chemokines, cytokines and reactive oxygen species are produced. J Neuroimmunol, 1999, 93, 182-193.
70. Bamberger, M. E.; Harris, M. E.; McDonald, D. R.; Husemann, J.; Landreth, G. E. A cell surface receptor complex for fibrillar beta-amyloid mediates microglial activation. J Neurosci, 2003, 23, 2665-2674.
71. Sapp, E.; Kegel, K. B.; Aronin, N.; Hashikawa, T.; Uchiyama, Y.; Tohyama, K.; Bhide, P. G.; Vonsattel, J. P.; DiFiglia, M. Early and progressive accumulation of reactive microglia in the Huntington disease brain. J Neuropathol Exp Neurol, 2001, 60, 161-172.
72. Wang, J. Y.; Shum, A. Y.; Ho, Y. J.; Wang, J. Y. Oxidative neurotoxicity in rat cerebral cortex neurons: synergistic effects of H202 and NO on apoptosis involving activation of p38 mitogen-activated protein kinase and caspase-3. J Neurosci Res, 2003, 72, 508-19.
73. Donnelly, S. Why is erythropoietin made in the kidney? The kidney functions as a critmeter. Am J Kidney Dis, 2001, 38, 415-425.
74. Gurney, A. M. Multiple sites of oxygen sensing and their contributions to hypoxic pulmonary vasoconstriction. Respir Physiol Neurobiol, 2002, 132, 43-53.
75. Fyhrquist, F.; Gronhagen-Riska, C.; Hortling, L.; Forslund, T.; Tikkanen, I. Regulation of angiotensin converting enzyme. J Hypertens Suppl, 1983, 1, 25-30.
76. Larsson, K.; Malmberg, P.; Eklund, A.; Belin, L.; Blaschke, E. Exposure to microorganisms, airway inflammatory changes and immune reactions in asymptomatic dairy farmers. Bronchoalveolar lavage evidence of macrophage activation and permeability changes in the airways. Int Arch Allergy Appl Immunol, 1988, 87, 127-133.
77. Bermudez, L. E.; Wu, M.; Young, L. S. Effect of stress-related hormones on macrophage receptors and response to tumor necrosis factor. Lymphokine Res, 1990, 9,137-145.
78. Rodgers, K.; Xiong, S.; Espinoza, T.; Roda, N.; Maldonado, S.; dizerega, G. S. Angiotensin II increases host resistance to peritonitis. Clin Diagn Lab Immunol, 2000, 7, 635-640.
79. Haznedaroglu, I. C.; Ozturk, M. A. Towards the understanding of the local hematopoietic bone marrow renin-angiotensin system. Int J Biochem Cell Biol, 2003, 35, 867-880.
80. Rodgers, K. E.; Xiong, S.; diZerega, G. S. Accelerated recovery from irradiation injury by angiotensin peptides. Cancer Chemother Pharmacol, 2002, 49, 403-411.
81. Hori, Y.; Wada, H.; Mori, Y.; Shimura, M.; Hiyoyama, K.; Nakasaki, T.; Nishii, K.; Tamaki, S.; Nishikawa, M.; Deguchi, K.; Minami, N.; Shiku, H. Plasma sFas and sFas ligand levels in patients with thrombotic thrombocytopenic purpura and in those with disseminated intravascular coagulation. Am J Hematol, 1999, 61, 21-25.
82. Moskowitz, D. W. Is Angiotensin I-Converting Enzyme a “Master” Disease Gene?Diabetes Technology & Therapeutics 2002, 4, 683-711.
83. McCully, K. S. Chemical Pathology of Homocysteine. I. Atherogenesis. Ann Clin Lab Sci., 1993, 23, 477-93.
84. McCully, K. S. Chemical Pathology of Homocysteine. II. Carcinogenesis and Homocysteine Thiolactone Metabolism. Ann Clin Lab Sci., 1994, 24, 27-59.
85. Le Noble, F. A.; Hekking, J. W.; Van Straaten, H. W.; Slaaf, D. W.; Struyker Boudier, H. A. Angiotensin II stimulates angiogenesis in the chorio-allantoic membrane of the chick embryo. Eur J Pharmacol, 1991, 195, 305-306.
86. Yoshida, K.; Yasujima, M.; Kohzuki, M.; Tsunoda, K.; Kudo, K.; Kanazawa, M.; Yabe, T.; Abe, K.; Yoshinaga, K. Chronic synergistic effect of endothelin-1 and angiotensin II on blood pressure in conscious rats. J Cardiovasc Pharmacol, 1991,17 Suppl 7, S514-S516.
87. Haver, V. M.; Namm, D. H. Generation of a vasoactive substance in human plasma during coagulation. Evidence of thrombin-induced contraction of rabbit aorta and dog coronary artery. Blood Vessels, 1983, 20, 92-98.
88. Antonaccio, M. J.; Normandin, D.; Serafino, R.; Moreland, S. Effects of thrombin and thrombin receptor activating peptides on rat aortic vascular smooth muscle. J Pharmacol Exp Ther, 1993, 266,125-132.
89. Fisslthaler, B.; Schini-Kerth, V. B.; Fleming, I.; Busse, R. Thrombin receptor expression is increased by angiotensin II in cultured and native vascular smooth muscle cells. Cardiovasc Res, 1998, 38, 263-271.
90. Fukuhara, M.; Neves, L. A.; Li, P.; Diz, D. I.; Ferrario, C. M.; Brosnihan, K. B. The angiotensin II ATI receptor antagonist irbesartan prevents thromboxane A2-induced vasoconstriction in the rat hind-limb vascular bed in vivo. J Hypertens, 2001, 19, 561-566.
91. Laher, L; Thompson, L. P.; Gagne, L. Protein kinase C as a modulator of response amplification in vascular smooth muscle. Blood Vessels, 1990, 27, 333-340.
92. Vaziri, N. D.; Zhou, X. J.; Smith, J.; Oveisi, F.; Baldwin, K.; Purdy, R. E. In vivo and in vitro pressor effects of erythropoietin in rats. Am J Physiol, 1995, 269, F838-F845.
93. Camussi, G.; Aglietta, M.; Malavasi, F.; Tefta, C.; Piacibello, W.; Sanavio, F.; Bussolino, F. The release of platelet-activating factor from human endothelial cells in culture. J Immunol, 1983, 131, 2397-2403.
94. Ishihara, N.; Sakamoto, H.; Iwarna, M.; Kobayashi, B. Net increase of platelet membrane tyrosine specific-protein kinase activity by phorbol myristate acetate. Life Sci, 1990, 46, 29-36.
95. Touyz, R. M.; Schiffrin, E. L. Effects of angiotensin II and endothelin-1 on platelet aggregation and cytosolic pH and free Ca2+ concentrations in essential hypertension. Hypertension, 1993, 22, 853-862.
96. Larsson, P. T.; Schwieler, J. H.; Wallen, N. H. Platelet activation during angiotensin II infusion in healthy volunteers. Blood Coagul Fibrinolysis, 2000, 11, 61-69.
97. Schwemmer, M.; Sommer, O.; Bassenge, E. Angiotensin receptor blocker losartan suppresses platelet activity by interfering with thromboxane signaling. Cardiovasc Drugs Ther, 2001, 15, 301-307.
98. Tamarat, R.; Silvestre, J. S.; Durie, M.; Levy, B. I. Angiotensin II angiogenic effect in vivo involves vascular endothelial growth factor- and inflammation-related pathways. Lab Invest, 2002, 82, 747-756.
99. Taylor, D. S.; Cheng, X.; Pawlowski, J. E.; Wallace, A. R.; Ferrer, P.; Molloy, C. J. Epiregulin is a potent vascular smooth muscle cell-derived mitogen induced by angiotensin II, endothelin-1, and thrombin. Proc Nat Acad Sci USA, 1999, 96, 1633-1638.
100. Powell, J. S.; Muller, R. K.; Rouge, M.; Kuhn, H.; Hefti, F.; Baumgartner, H. R. The proliferative response to vascular injury is suppressed by angiotensin-converting enzyme inhibition. J Cardiovasc Pharmacol, 1990, 16 Suppl 4, S42-S49.
101. Costanzo, A.; Moretti, F.; Burgio, V. L.; Bravi, C.; Guido, F.; Levrero, M.; Puri, P. L. Endothelial activation by angiotensin II through NFkappaB and p38 pathways: Involvement of NFkappaB-inducible kinase (NIK), free oxygen radicals, and selective inhibition by aspirin. J Cell Physiol, 2003, 195, 402-410.
102. Suzuki, J.; Iwai, M.; Nakagami, H.; Wu, L.; Chen, R.; Sugaya, T.; Hamada, M.; Hiwada, K.; Horiuchi, M. Role of angiotensin II-regulated apoptosis through distinct AT1 and AT2 receptors in neointimal formation. Circulation, 2002, 106, 847-853.
103. Wang, R.; Zagariya, A.; Ibarra-Sunga, O.; Gidea, C.; Ang, E.; Deshmukh, S.; Chaudhary, G.; Baraboutis, J.; Filippatos, G.; Uhal, B. D. Angiotensin II induces apoptosis in human and rat alveolar epithelial cells. Am J Physiol, 1999, 276, L885-L889.
104. Yamada, T.; Horiuchi, M.; Dzau, V. J. Angiotensin II type 2 receptor mediates programmed cell death. Proc Natl Acad Sci USA, 1996, 93, 156-160.
105. Furchgott R F. Introduction to EDRF research. J Cardiovasc Pharmacol, 1993, 22 Suppl 7, S1-S2.
106. Trulsson, L. M.; Gasslander, T.; Sundqvist, T.; Svanvik, J. The influence of nitric oxide on basal and cholecystokinin-8-induced proliferation and apoptosis in the rat pancreas. Regul Pept, 2002, 106, 97-104.
107. Gibbons G H. Vasculoprotective and cardioprotective mechanisms of angiotensin-converting enzyme inhibition: the homeostatic balance between angiotensin II and nitric oxide. Clin Cardiol, 1997, 20(11 Suppl 2), II-18-25.
108. Millatt, L. J.; Abdel-Rahman, E. M.; Siragy, H. M. Angiotensin II and nitric oxide: a question of balance. Regul Pept, 1999, 81, 1-10.
109. White, C. R.; Darley-Usmar, V.; Berrington, W. R.; McAdams, M.; Gore, J. Z.; Thompson, J. A.; Parks, D. A.; Tarpey, M. M.; Freeman, B. A. Circulating plasma xanthine oxidase contributes to vascular dysfunction in hypercholesterolemic rabbits. Proc Natl Acad Sci USA, 1996, 93, 8745-8749.
110. Rajagopalan, S.; Kurz, S.; Munzel, T.; Tarpey, M.; Freeman, B. A.; Griendling, K. K.; Harrison, D. G. Angiotensin II-mediated hypertension in the rat increases vascular superoxide production via membrane NADH/NADPH oxidase activation. Contribution to alterations of vasomotor tone. J Clin Invest, 1996, 97, 1916-1923.
111. Treasure, C. B.; Klein, J. L.; Vita, J. A.; Manoukian, S. V.; Renwick, G. H.; Selwyn, A. P.; Ganz, P.; Alexander, R. W. Hypertension and left ventricular hypertrophy are associated with impaired endothelium-mediated relaxation in human coronary resistance vessels. Circulation, 1993, 87, 86-93.
112. Antony, I.; Lerebours, G.; Nitenberg, A. Angiotensin-converting enzyme inhibition restores flow-dependent and cold pressor test-induced dilations in coronary arteries of hypertensive patients. Circulation, 1996, 94, 3115-3122.
113. Donnelly, S. M.; Miller, J. A. Losartan may modulate erythropoietin production. J Renin Angiotensin Aldosterone Syst, 2001, 2, 255-260.
114. Wang, A. Y.; Yu, A. W.; Lam, C. W.; Yu, L. M.; Li, P. K.; Goh, J.; Lui, S. F. Effects of losartan or enalapril on hemoglobin, circulating erythropoietin, and insulin-like growth factor-1 in patients with and without posttransplant erythrocytosis. Am J Kidney Dis, 2002, 39, 600-608.
115. Cole, J.; Ertoy, D.; Lin, H.; Sutliff, R. L.; Ezan, E.; Guyene, T. T.; Capecchi, M.; Corvol, P.; Bernstein, K. E. Lack of angiotensin II-facilitated erythropoiesis causes anemia in angiotensin-converting enzyme-deficient mice. J Clin Invest, 2000, 106, 1391-1398.
116. Chan, P. C.; Wei, D. C.; Tam, S. C.; Chan, F. L.; Yeung, W. C.; Cheng, I. K. Post-transplant erythrocytosis: role of erythropoietin and male sex hormones. Nephrol Dial Transplant, 1992, 7, 137-142.
117. Islam, M. S.; Bourbigot, B.; Codet, J. P.; Songy, B.; Fournier, G.; Cledes, J. Captopril induces correction of postrenal transplant erythremia. Transpl Int, 1990, 3, 222-225.
118. Midtvedt, K.; Stokke, E. S.; Hartmann, A. Successful long-term treatment of post-transplant erythrocytosis with losartan. Nephrol Dial Transplant, 1996, 11, 2495-2497.
119. Silber S J. Growth of baby kidneys transplanted into adults. Arch Surg, 1976, 111, 75-77.
120. Semenza, G. L. Regulation of erythropoietin production. New insights into molecular mechanisms of oxygen homeostasis. Hematol Oncol Clin North Am, 1994, 8, 863-884.
121. Richard, D. E.; Berra, E.; Pouyssegur, J. Nonhypoxic pathway mediates the induction of hypoxia-inducible factor 1alpha in vascular smooth muscle cells. J Biol Chem, 2000, 275, 26765-26771.
122. Bilton, R. L.; Booker, G. W. The subtle side to hypoxia inducible factor (HIFalpha) regulation. Eur J Biochem, 2003, 270, 791-798.
123. McMahon, T. J.; Moon, R. E.; Luschinger, B. P.; Carraway, M. S.; Stone, A. E.; Stolp, B. W.; Gow, A. J.; Pawloski, J. R.; Watke, P.; Singel, D. J.; Piantadosi, C. A.; Stamler, J. S. Nitric oxide in the human respiratory cycle. Nat Med, 2002, 8, 711-717.
124. Lane, P.; Gross, S. Hemoglobin as a chariot for NO bioactivity. Nat Med, 2002, 8, 657-658.
125. Lane P, Hao G, Gross S S. S-nitrosylation is emerging as a specific and fundamental posttranslational protein modification: head-to-head comparison with O-phosphorylation. Sci STKE, 2001, 2001, RE1.
126. Pawloski, J. R.; Hess, D. T.; Stamler, J. S. Export by red blood cells of nitric oxide bioactivity. Nature, 2001, 409, 622-626.
127. Rafikova, O.; Rafikov, R.; Nudler, E. Catalysis of S-nitrosothiols formation by serum albumin: the mechanism and implication in vascular control. Proc Natl Acad Sci USA, 2002, 99, 5913-5918.
128. Joles, J. A.; Stroes, E. S.; Rabelink, T. J. Endothelial function in proteinuric renal disease. Kidney Int Suppl, 1999, 71, S57-S61.
129. Niederberger, M.; Schrier, R. W. Pathogenesis of sodium and water retention in liver disease. Prog Liver Dis, 1992, 10, 329-347.
130. Kurtzman, N. A. Nephritic edema. Semin Nephrol, 2001, 21, 257-261.
131. Dakic, A.; Wu, L. Hemopoietic precursors and development of dendritic cell populations. Leuk Lymphoma, 2003, 44,1469-1475.
132. Luft, F. C. [Innate and acquired immunity in angiotensin-induced malignant hypertension] Verh Dtsch Ges Pathol, 2002, 86, 76-82.
133. Muller, D. N.; Shagdarsuren, E.; Park, J. K.; Dechend, R.; Mervaala, E.; Hampich, F.; Fiebeler, A.; Ju, X.; Finckenberg, P.; Theuer, J.; Viedt, C.; Kreuzer, J.; Heidecke, H.; Haller, H.; Zenke, M.; Luft, F. C. Immunosuppressive treatment protects against angiotensin II-induced renal damage. Am J Pathol, 2002, 161,1679-1693.
134. Dandona, P.; Kumar, V.; Aljada, A.; Ghanim, H.; Syed, T.; Hofinayer, D.; Mohanty, P.; Tripathy, D.; Garg, R. Angiotensin II receptor blocker valsartan suppresses reactive oxygen species generation in leukocytes, nuclear factor-kappa B, in mononuclear cells of normal subjects: evidence of an antiinflammatory action. J Clin Endocrinol Metab, 2003, 88, 4496-4501.
135. Gaur, U.; Aggarwal, B. B. Regulation of proliferation, survival and apoptosis by members of the TNF superfamily. Biochem Pharmacol, 2003, 66,1403-1408.
136. Guergnon, J.; Chaussepied, M.; Sopp, P.; Lizundia, R.; Moreau, M. F.; Blumen, B.; Werling, D.; Howard, C. J.; Langsley, G. A tumour necrosis factor alpha autocrine loop contributes to proliferation and nuclear factor-kappaB activation of Theileria parva-transformed B cells. Cell Microbiol, 2003, 5, 709-716.
137. Theuer, J.; Dechend, R.; Muller, D. N.; Park, J. K.; Fiebeler, A.; Barta, P.; Ganten, D.; Haller, H.; Dietz, R.; Luft, F. C. Angiotensin II induced inflammation in the kidney and in the heart of double transgenic rats. BMC Cardiovasc Disord, 2002, 2, 3.
138. Wu, L. L.; Yang, N.; Roe, C. J.; Cooper, M. E.; Gilbert, R. E.; Atkins, R. C.; Lan, H. Y. Macrophage and myofibroblast proliferation in remnant kidney: role of angiotensin II. Kidney Int Suppl, 1997, 63, S221-S225.
139. Cogolludo, A.; Moreno, L.; Bosca, L.; Tamargo, J.; Perez-Vizcaino, F. Thromboxane A2-induced inhibition of voltage-gated K+ channels and pulmonary vasoconstriction: role of protein kinase Czeta. Circ Res, 2003, 93, 656-663.
140. Marshall, R. P.; McAnulty, R. J.; Laurent, G. J. Angiotensin II is mitogenic for human lung fibroblasts via activation of the type 1 receptor. Am J Respir Crit Care Med, 2000, 161, 1999-2004.
141. Lam, S. Y.; Fung, M. L.; Leung, P. S. Regulation of the angiotensinconverting enzyme activity by a time-course hypoxia in the carotid body. J Appl Physiol, 2003, Oct 3 [Epub ahead of print]
142. Tuder, R. M.; Petrache, I.; Elias, J. A.; Voelkel, N. F.; Henson, P. M. Apoptosis and emphysema: the missing link. Am J Respir Cell Mol Biol, 2003, 28, 551-554.
143. Kawakami, Y.; Yamaguchi, E.; Munakata, M.; Dosaka-Akita, H.; Furuya, K. Genetic factors in lung disease. Part 11: Lung cancer and angiotensin converting enzyme gene. Respirology, 1997, 2, 81-90. Erratum in: Respirology, 1998, 3, 61.
144. Alho, H. S.; Maasilta, P. K.; Harjula, A. L.; Hammainen, P.; Salminen, J.; Salminen, U.S. Tumor necrosis factor-alpha in a porcine bronchial model of obliterative bronchiolitis. Transplantation, 2003, 76, 516-523.
145. Jaffe, A.; Balfour-Lynn, I. M. Treatment of severe small airways disease in children with cystic fibrosis: alternatives to corticosteroids. Paediatr Drugs, 2002, 4, 381-389.
146. Raiden, S.; Nahmod, K.; Nahmod, V.; Semeniuk, G.; Pereira, Y.; Alvarez, C.; Giordano, M.; Geffner, J. R. Nonpeptide antagonists of ATI receptor for angiotensin II delay the onset of acute respiratory distress syndrome. J Pharmacol Exp Ther, 2002, 303, 45-51.
147. Moskowitz, D. W. Why ATIR Blockade Makes Sense for SARS. http://www.genomedics.com/investor/geno.pdf
148. Wang, L. W.; Fu, X. L.; Clough, R.; Sibley, G.; Fan, M.; Bentei, G. C.; Marks, L. B.; Anscher, M. S. Can angiotensin-converting enzyme inhibitors protect against symptomatic radiation pneumonitis? Radiat Res, 2000, 153, 405-410.
149. Yang, G.; Madan, A.; Dennery, P. A. Maturational differences in hyperoxic AP-1 activation in rat lung. Am J Physiol Lung Cell Mol Physiol, 2000, 278, L393-L398.
150. Hayward, L. D.; Angyal, S. J. A Symmetry rule for the Circular Dichroism of Reducing Sugars, and the Proportion of Carbonyl Forms in Aqueous Solutions Thereof, Carbohydr. Res., 1977, 53, 13-20.
151. Nathan, D. M.; Lachin, J.; Cleary, P.; Orchard, T.; Brillon, D. J.; Backlund, J. Y.; O'Leary, D. H.; Genuth, S. Diabetes Control and Complications Trial; Epidemiology of Diabetes Interventions and Complications Research Group. Intensive diabetes therapy and carotid intima-media thickness in type 1 diabetes mellitus. N Engl. J Med, 2003, 348, 2294-2303.
152. Derubertis, F. R.; Craven, P. A. Activation of Protein Kinase C in Glomerular Cells in Diabetes. Mechanisms and Potential Links to the Pathogenesis of Diabetic Glomerulopathy. Diabetes, 1994, 43, 1-8.
153. Lindschau, C.; Quass, P.; Menne, J.; Guler, F.; Fiebeler, A.; Leitges, M.; Luft, F. C.; Haller, H. Glucose-induced TGF-betal and TGF-beta Receptor-1 Expression in Vascular Smooth Muscle Cells is Mediated by Protein Kinase C-alpha. Hypertension, 2003, 42, 335-41.
154. Takayama, S.; White, M. F.; Kahn, C. R. Phorbol ester-induced serine phosphorylation of the insulin receptor decreases its tyrosine kinase activity. J Biol Chem, 1988, 263, 3440-3447.
155. Ustundag, B.; Cay, M.; Naziroglu, M.; Dilsiz, N.; Crabbe, M. J.; Ilhan, N. The study of renin-angiotensin-aldosterone in experimental diabetes mellitus. Cell Biochem Funct, 1999, 17, 193-198.
156. Okada, Y.; Yamanaka, I.; Sakamoto, T.; Hata, Y.; Sassa, Y.; Yoshikawa, H.; Fujisawa, K.; Ishibashi, T.; Inomata, H. Increased expression of angiotensin-converting enzyme in retinas of diabetic rats. Jpn J Ophthalmol, 2001, 45, 585-591.
157. Hara, K.; Kobayashi, N.; Watanabe, S.; Tsubokou, Y.; Matsuoka, H. Effects of quinapril on expression of eNOS, ACE, and ATI receptor in deoxycorticosterone acetate-salt hypertensive rats. Am J Hypertens, 2001, 14, 321-330.
158. Hanefeld, M.; Kohler, C. [The metabolic syndrome and its epidemiologic dimensions in historical perspective] Z Arztl Fortbild Qualitatssich, 2002, 96, 183-188.
159. Johnson, R. J.; Kang, D. H.; Feig, D.; Kivlighn, S.; Kanellis, J.; Watanabe, S.; Tuttle, K. R.; Rodriguez-Iturbe, B.; Herrera-Acosta, J.; Mazzali, M. Is there a pathogenetic role for uric acid in hypertension and cardiovascular and renal disease? Hypertension, 2003, 41, 1183-1190.
160. Bachschmid, M.; Thurau, S.; Zou, M. H.; Ullrich, V. Endothelial cell activation by endotoxin involves superoxide/NO-mediated nitration of prostacyclin synthase and thromboxane receptor stimulation. FASEB J, 2003, 17, 914-916.
161. Mazzali, M.; Hughes, J.; Kim, Y. G.; Jefferson, J. A.; Kang, D. H.; Gordon, K. L.; Lan, H. Y.; Kivlighn, S.; Johnson, R. J. Elevated uric acid increases blood pressure in the rat by a novel crystal-independent mechanism. Hypertension, 2001, 38, 1101-1106.
162. Brown, N. J.; Vaughan, D. E. Prothrombotic effects of angiotensin. Adv Intern Med, 2000, 45, 419-429.
163. Perez-Ruiz, A.; Montes, R.; Velasco, F.; Lopez-Pedrera, C.; Antonio-Paramo, J.; Orbe, J.; Hermida, J.; Rocha, E. Regulation by nitric oxide of endotoxin-induced tissue factor and plasminogen activator inhibitor-1 in endothelial cells. Thromb Haemost, 2002, 88, 1060-1065.
164. Tedgui, A.; Mallat, Z. Apoptosis, a major determinant of atherothrombosis. Arch Mal Coeur Vaiss, 2003, 96, 671-675.
165. Belhassen, L.; Pelle, G.; Sediame, S.; Bachir, D.; Carville, C, Bucherer C, Lacombe C, Galacteros F, Adnot S. Endothelial dysfunction in patients with sickle cell disease is related to selective impairment of shear stress-mediated vasodilation. Blood, 2001, 97, 1584-1589.
166. Barabino G A, McIntire L V, Eskin S G, Sears D A, Udden M. Rheological studies of erythrocyte-endothelial cell interactions in sickle cell disease. Prog Clin Biol Res, 1987, 240, 113-127.
167. Reiter, C. D.; Wang, X.; Tanus-Santos, J. E.; Hogg, N.; Cannon, R. O. 3rd; Schechter, A X; Gladwin, M. T. Cell-free hemoglobin limits nitric oxide bioavailability in sickle-cell disease. Nat Med, 2002, 8, 1383-1389.
168. Bouba, L; Makrydimas, G.; Kalaitzidis, R.; Lolis, D. E.; Siamopoulos, K. C.; Georgiou, I. Interaction between the polymorphisms of the reninangiotensin system in preeclampsia. Eur J Obstet Gynecol Reprod Biol, 2003, 110, 8-11.
169. Teragawa, H.; Matsuura, H.; Chayama, K.; Oshima, T. Mechanisms responsible for vasodilation upon magnesium infusion in vivo: clinical evidence. Magnes Res, 2002, 15, 241-246.
170. Salzet, M.; Verger-Bocquet, M. Elements of angiotensin system are involved in leeches and mollusks immune response modulation. Brain Res Mol Brain Res, 2001, 94, 137-147.
171. Watanabe, T.; Saiki, Y.; Sakata, Y. The effect of central angiotensin II receptor blockade on interleukin-1 beta- and prostaglandin E-induced fevers in rats: possible involvement of brain angiotensin II receptor in fever induction. J Pharmacol Exp Ther, 1997, 282, 873-881.
172. Balyasnikova, I V, Metzger R, Franke F E, Danilov S M. Monoclonal antibodies to denatured human ACE (CD 143), broad species specificity, reactivity on paraffin sections, and detection of subtle conformational changes in the C-terminal domain of ACE. Tissue Antigens, 2003, 61, 49-62.
173. Costerousse, O.; Allegrini, J.; Lopez, M.; Alhenc-Gelas, F. Angiotensin 1-converting enzyme in human circulating mononuclear cells: genetic polymorphism of expression in T-lymphocytes. Biochem J, 1993, 290, 33-40.
174. Rohrbach, M. S.; Conrad, A. K. Comparison of the T lymphocyte-dependent induction of angiotensin-converting enzyme and leucine aminopeptidase in cultured human monocytes. Clin Exp Immunol, 1991, 83, 510-515.
175. Villard, E.; Alonso, A.; Agrapart, M.; Challah, M.; Soubrier, F. Induction of angiotensin I-converting enzyme transcription by a protein kinase C-dependent mechanism in human endothelial cells. J Biol Chem, 1998, 273, 25191-25197.
176. Hahn, A. W.; Jonas, U.; Buhler, F. R.; Resink, T. J. Activation of human peripheral monocytes by angiotensin II. FEBS Lett, 1994, 347, 178-180.
177. Rice, E. K.; Tesch, G. H.; Cao, Z.; Cooper, M. E.; Metz, C. N.; Bucala, R.; Atkins, R. C.; Nikolic-Paterson, D. J. Induction of MIF synthesis and secretion by tubular epithelial cells: a novel action of angiotensin II. Kidney Int, 2003, 63, 1265-1275.
178. Sato, H.; Watanabe, A.; Tanaka, T.; Koitabashi, N.; Arai, M.; Kurabayashi, M.; Vokoyama, T. Regulation of the human tumor necrosis factor-alpha promoter by angiotensin II and lipopolysaccharide in cardiac fibroblasts: different cis-acting promoter sequences and transcriptional factors. J Mol Cell Cardiol, 2003, 35, 1197-1205.
179. Amann, B.; Tinzmann, R.; Angelkort, B. ACE inhibitors improve diabetic nephropathy through suppression of renal MCP-1. Diabetes Care, 2003, 26, 2421-2425.
180. Femandez-Castelo, S.; Arzt, E. S.; Pesce, A.; Criscuolo, M. E.; Diaz, A.; Finkielman, S.; Nahmod, V. E. Angiotensin II regulates interferon-gamma production. J Interferon Res, 1987, 7, 261-268.
181. Horiuchi, M.; Akishita, M.; Dzau, V. J. Molecular and cellular mechanism of angiotensin II-mediated apoptosis. Endocr Res, 1998, 24, 307-314.
182. Tanaka, A.; Matsumori, A.; Wang, W.; Sasayama, S. An angiotensin II receptor antagonist reduces myocardial damage in an animal model of myocarditis. Circulation, 1994, 90, 2051-2055.
183. Martin, M. F.; Surrall, K. E.; McKenna, F.; Dixon, J. S.; Bird, H. A.; Wright, V. Captopril: a new treatment for rheumatoid arthritis? Lancet, 1984, 1, 1325-1328.
184. Prkacin, I.; Novak, B.; Sertic, J.; Mrzljak, A. Angiotensin-converting enzyme gene polymorphism in patients with systemic lupus. Acta Med Croatica, 2001, 55, 73-76.
185. Lieberman, J.; Bell, D. S. Serum angiotensin-converting enzyme as a marker for the chronic fatigue-immune dysfunction syndrome: a comparison to serum angiotensin-converting enzyme in sarcoidosis. Am J Med, 1993, 95, 407-412.
186. Cheng, S.; Schmidt-Grimminger, D. C.; Murant, T.; Broker, T. R.; Chow, L. T. Differentiation-dependent up-regulation of the human papillomavirus E7 gene reactivates cellular DNA replication in suprabasal differentiated keratinocytes. Genes Dev, 1995, 9, 2335-2349.
187. Thomas, D. W.; Hoffman, M. D. Identification of macrophage receptors for angiotensin: a potential role in antigen uptake for T lymphocyte responses? J Immunol, 1984, 132, 2807-2812.
188. Crowe, S. M. Role of macrophages in the pathogenesis of human immunodeficiency virus (HIV) infection. Aust NZ J Med, 1995, 25, 777-783.
189. Higuchi, H.; Gores, G. J. Mechanisms of liver injury: an overview. Curr Mol Med, 2003, 3, 483-490.
190. Bataller, R.; Gabele, E.; Schoonhoven, R.; Morris, T.; Lehnert, M.; Yang, L.; Brenner, D. A.; Rippe, R. A. Prolonged infusion of angiotensin II into normal rats induces stellate cell activation and proinflammatory events in liver. Am J Physiol Gastrointest Liver Physiol, 2003, 285, G642-G651.
191. Bataller, R.; Sancho-Bru, P.; Gines, P.; Lora, J. M.; Al-Garawi, A.; Sole, M.; Colmenero, J.; Nicolas, J. M.; Jimenez, W.; Weich, N.; Gutierrez-Ramos, J. C.; Arroyo, V.; Rodes, J. Activated human hepatic stellate cells express the renin-angiotensin system and synthesize angiotensin II. Gastroenterology, 2003, 125, 117-125.
192. Kurikawa, N.; Suga, M.; Kuroda, S.; Yamada, K.; Ishikawa, H. An angiotensin II type 1 receptor antagonist, olmesartan medoxomil, improves experimental liver fibrosis by suppression of proliferation and collagen synthesis in activated hepatic stellate cells. Br J Pharmacol, 2003, 139, 1085-1094.
193. Yamada, T.; Kuno, A.; Masuda, K.; Ogawa, K.; Sogawa, M.; Nakamura, S.; Ando, T.; Sano, H.; Nakazawa, T.; Ohara, H.; Nomura, T.; Joh, T.; Itoh, M. Candesartan, an angiotensin II receptor antagonist, suppresses pancreatic inflammation and fibrosis in rats. J Pharmacol Exp Ther, 2003, 307, 17-23.
194. Li, H.; Pamukcu, R.; Thompson, W. J. beta-Catenin signaling: therapeutic strategies in oncology. Cancer Biol Ther, 2002, 1, 621-625.
195. Murray, N. R.; Davidson, L. A.; Chapkin, R. S.; Clay Gustafson, W.; Schattenberg, D. G.; Fields, A. P. Overexpression of protein kinase C betall induces colonic hyperproliferation and increased sensitivity to colon carcinogenesis. J Cell Biol, 1999, 145, 699-711.
196. Koh, W. P.; Yuan, J. M.; Sun, C. L.; van den Berg, D.; Seow, A.; Lee, H. P.; Yu, M. C. Angiotensin I-converting enzyme (ACE) gene polymorphism and breast cancer risk among Chinese women in Singapore. Cancer Res, 2003, 63, 573-578.
197. Abali, H.; Gullu, I. H.; Engin, H.; Haznedaroglu, I. C.; Erman, M.; Tekuzman, G. Old antihypertensives as novel antineoplastics: angiotensin-I-converting enzyme inhibitors and angiotensin II type 1 receptor antagonists. Med Hypotheses, 2002, 59, 344-348.
198. Yoshiji, H.; Kuriyama, S.; Fukui, H. Perindopril: possible use in cancer therapy. Anticancer Drugs, 2002, 13, 221-228.
199. Ishii, H.; Okada, S.; Nose, H.; Yoshimori, M.; Aoki, K.; Okusaka, T. Prognostic factors in patients with advanced pancreatic cancer treated with systemic chemotherapy. Pancreas, 1996, 12, 267-271.
200. Ueno, H.; Okada, S.; Okusaka, T.; Ikeda, M. Prognostic factors in patients with metastatic pancreatic adenocarcinoma receiving systemic chemotherapy. Oncology, 2000, 59, 296-301.
201. Nassis, L.; Frauman, A. G.; Ohishi, M.; Zhuo, J.; Casley, D. J.; Johnston, C. I.; Fabiani, M. E. Localization of angiotensin-converting enzyme in the human prostate: pathological expression in benign prostatic hyperplasia. J Pathol, 2001, 195, 571-579.
202. Bhatia-Gaur, R.; Donjacour, A. A.; Sciavolino, P. J.; Kim, M.; Desai, N.; Young, P.; Norton, C. R.; Gridley, T.; Cardiff, R. D.; Cunha, G. R.; Abate-Shen, C.; Shen, M. M. Roles for Nkx3.1 in prostate development and cancer. Genes Dev, 1999, 13, 966-977.
203. Wingender, E.; Chen, X.; Fricke, E.; Geffers, R.; Hehl, R.; Liebich, I.; Krull, M.; Matys, V.; Michael, H.; Ohnhauser, R.; Pruss, M.; Schacherer, F.; Thiele, S.; Urbach, S. The TRANSFAC system on gene expression regulation. Nucleic Acids Res, 2001, 29, 281-283.
204. Fabiani, M. E.; Hawkes, D. J.; Frauman, A. G.; Tikellis, C.; Johnston, C. I.; Wilkinson-Berka, J. L. Regulation of angiotensin II receptors in the prostate of the transgenic (mRen-2)27 rat: effect of angiotensin-converting enzyme inhibition. Int J Biochem Cell Biol, 2003, 35, 973-83.
205. Seidenfeld, J.; Samson, D. J.; Hasselblad, V.; Aronson, N.; Albertsen, P. C.; Bennett, C. L.; Wilt, T. J. Single-therapy androgen suppression in men with advanced prostate cancer: a systematic review and meta-analysis. Ann Intern Med, 2000, 132, 566-577.
206. Jaiswal, A. K.; Panda, J. N.; Kumar, M. V.; Joshi, P. Androgen dependence of testicular and epididymal angiotensin converting enzyme. Andrologia, 1985, 17, 92-97.
207. Freshour, J. R.; Chase, S. E.; Vikstrom, K. L. Gender differences in cardiac ACE expression are normalized in androgen-deprived male mice. Am J Physiol Heart Circ Physiol, 2002, 283, H1997-H2003.
208. Fabian, J. R.; Kane, C. M.; Abel, K. J.; Gross, K. W. Expression of the mouse Ren-1 gene in the coagulating gland: localization and regulation. Biol Reprod, 1993, 48, 1383-1394.
209. Abate-Shen, C.; Banach-Petrosky, W. A.; Sun, X.; Economides, K. D.; Desai, N.; Gregg, J. P.; Borowsky, A. D.; Cardiff, R. D.; Shen, M. M. Nkx3.1; Pten mutant mice develop invasive prostate adenocarcinoma and lymph node metastases. Cancer Res, 2003, 63, 3886-3890.
210. Abdulkadir, S. A.; Magee, J. A.; Peters, T. J.; Kaleem, Z.; Naughton, C. K.; Humphrey, P. A.; Milbrandt, J. Conditional loss of Nkx3.1 in adult mice induces prostatic intraepithelial neoplasia. Mol Cell Biol, 2002, 22, 1495-1503.
211. Oettgen, P.; Finger, E.; Sun, Z.; Akbarali, Y.; Thamrongsak, U.; Boltax, J.; Grall, F.; Dube, A.; Weiss, A.; Brown, L.; Quinn, G.; Kas, K.; Endress, G.; Kunsch, C.; Libermann, T. A. PDEF, a novel prostate epithelium-specific ets transcription factor, interacts with the androgen receptor and activates prostate-specific antigen gene expression. J Biol Chem, 2000, 275, 1216-1225.
212. Chen, H.; Nandi, A. K.; Li, X.; Bieberich, C. J. NKX-3.1 interacts with prostate-derived Ets factor and regulates the activity of the PSA promoter. Cancer Res, 2002, 62, 338-340.
213. Sato, N.; Sadar, M. D.; Bruchovsky, N.; Saatcioglu, F.; Rennie, P. S.; Sato, S.; Lange, P. H.; Gleave, M. E. Androgenic Induction of Prostate-specific Antigen Gene Is Repressed by Protein-Protein Interaction between the Androgen Receptor and AP-1/c-Jun in the Human Prostate Cancer Cell Line LNCaP. J Biol Chem, 1997, 272, 17485-17494.
214. Proudier, A. J.; Cooper, A.; Whitehead, M.; Stevenson, J. C. Effects of oestrogen-only and oestrogen-progestogen replacement therapy upon circulating angiotensin I-converting enzyme activity in postmenopausal women. Clin Endocrinol (Oxf), 2003, 58, 30-35.
215. Mcleod, D. G. Hormonal therapy: historical perspective to future directions. Urology, 2003, 61, 3-7.
216. Pias, E. K.; Aw, T. Y. Apoptosis in mitotic competent undifferentiated cells is induced by cellular redox imbalance independent of reactive oxygen species production. FASEB J, 2002, 16, 781-790.
217. Moosmann, B.; Behl, C. Antioxidants as treatment for neurodegenerative disorders. Expert Opin Investig Drugs, 2002, 11, 1407-1435.
218. Mattson, M. P.; Sherman, M. Perturbed signal transduction in neurodegenerative disorders involving aberrant protein aggregation. Neuromolecular Med, 2003, 4, 109-132.
219. Carella, G. Introduction to apoptosis in ophthalmology. Eur J Ophthalmol, 2003, 13 Suppl 3, S5-S10.
220. Cameron, N. E.; Eaton, S. E.; Cotter, M. A.; Tesfaye, S. Vascular factors and metabolic interactions in the pathogenesis of diabetic neuropathy. Diabetologia, 2001, 44, 1973-1988.
221. Zochodne, D. W. Nerve and ganglion blood flow in diabetes: an appraisal. Int Rev Neurobiol, 2002, 50,161-202.
222. Schmeichel, A. M.; Schmeizer, J. D.; Low, P. A. Oxidative injury and apoptosis of dorsal root ganglion neurons in chronic experimental diabetic neuropathy. Diabetes, 2003, 52, 165-171.
223. Feldman, E. L. Oxidative stress and diabetic neuropathy: a new understanding of an old problem. J Clin Invest, 2003, 111, 431-433.
224. Malik, R. A. Can diabetic neuropathy be prevented by angiotensin-converting enzyme inhibitors? Ann Med, 2000, 32, 1-5.
225. Aggarwal, M.; Singh, J.; Sood, S.; Arora, B. Effects of lisinopril on streptozotocin-induced diabetic neuropathy in rats. Methods Find Exp Clin Pharmacol, 2001, 23, 131-134.
226. Munzel, T.; Keaney, J. F. Jr. Are ACE inhibitors a “magic bullet” against oxidative stress? Circulation, 2001, 104, 1571-1574.
227. Wang E. Regulation of apoptosis resistance and ontogeny of age-dependent diseases. Exp Gerontol, 1997, 32, 471-84.
228. Kirkwood, T. B. The origins of human ageing. Philos Trans R Soc Lond B Biol Sci, 1997, 352, 1765-1772.
229. Partridge, L.; Gems, D. Mechanisms of ageing: public or private? Nat Rev Genet, 2002, 3, 165-175.
230. Holliday, R. Food, reproduction and longevity: is the extended lifespan of calorie-restricted animals an evolutionary adaptation? Bioessays, 1989, 10, 125-127.
231. de Souza-Pinto, N. C.; Bohr, V. A. The mitochondrial theory of aging: involvement of mitochondrial DNA damage and repair. Int Rev Neurobiol, 2002, 53, 519-534.
232. Cho, C. G.; Kim, H. J.; Chung, S. W.; Jung, K. J.; Shim, K. H.; Yu, B. P.; Yodoi, J.; Chung, H. Y. Modulation of glutathione and thioredoxin systems by calorie restriction during the aging process. Exp Gerontol, 2003, 38, 539-548.
233. Norman, J. T.; Stidwill, R.; Singer, M.; Fine, L. G. Angiotensin II blockade augments renal cortical microvascular p02 indicating a novel, potentially renoprotective action. Nephron Physiol, 2003, 94, 39-46.
234. Bobyleva-Guarriero, V.; Wehbie, R. S.; Lardy, H. A. The role of malate in hormone-induced enhancement of mitochondrial respiration. Arch Biochem Biophys, 1986, 245, 477-482.
235. Mazzocchi, G.; Robba, C.; Rebuffat, P.; Nussdorfer, G. G. Investigations on the turnover of adrenocortical mitochondria. XV. A stereological study of the effect of chronic treatment with angiotensin II on the size and number of the mitochondria in the zona glomerulosa of the rat. Cell Tissue Res, 1980, 210, 333-337.
236. de Cavanagh, E. M.; Piotrkowski, B.; Basso, N.; Stella, I.; Inserra, F.; Ferder, L.; Fraga, C. G. Enalapril and losartan attenuate mitochondrial dysfunction in aged rats. FASEB J, 2003, 17, 1096-1098.
237. Tatar, M.; Kopelman, A.; Epstein, D.; Tu, M. P.; Yin, C. M.; Garofalo, R. S. A mutant Drosophila insulin receptor homolog that extends life-span and impairs neuroendocrine function. Science, 2001, 292, 107-110.
238. Hasty, P.; Campisi, J.; Hoeijmakers, J.; van Steeg, H.; Vijg, J. Aging and genome maintenance: lessons from the mouse? Science, 2003, 299, 1355-1359.
239. Cong, Y. S.; Wright, W. E.; Shay, J. W. Human telomerase and its regulation. Microbiol Mol Biol Rev, 2002, 66, 407-425.
240. Bonnet F, Cao Z, Cooper M E. Apoptosis and angiotensin II: yet another renal regulatory system? Exp Nephrol, 2001, 9, 295-300.
241. Brink, M.; Price, S. R.; Chrast, J.; Bailey, J. L.; Anwar, A.; Mitch, W. E.; Delafontaine, P. Angiotensin II induces skeletal muscle wasting through enhanced protein degradation and down-regulates autocrine insulin-like growth factor 1. Endocrinology, 2001, 142, 1489-1496.
242. Brink, M.; Anwar, A.; Delafontaine, P. Neurohormonal factors in the development of catabolic/anabolic imbalance and cachexia. Int J Cardiol, 2002, 85, 111-124.
243. Vescovo, G.; Ambrosio, G. B.; Daila Libera, L. Apoptosis and changes in contractile protein pattern in the skeletal muscle in heart failure. Acta Physiol Scand, 2001, 171, 305-310.

### BRIEF DESCRIPTION OF THE FIGURES

FIG. 1 depicts a ribbon model of tACE with an inhibitor bound to the active site.

FIG. 2 depicts tACE as redox- and mechanosensor.

FIG. 3 depicts In silico reduction of tACE.

FIG. 4a depicts sACE.

FIG. 4b depicts sACE showing cmplementarity of the N— and Cterminal domains.

FIG. 5 depicts the disulfide zipper of sACE.

FIG. 6 depicts the disulfide isomerase exchange reaction possible at the heart of the disulfide zupper.

FIG. 7 depicts the two steps in the catalytic cycle of the reciprocating enzyme, sACE.

FIG. 8 depicts the pathway for transfer of NO from oxygenated hemoglobin to sACE.

FIG. 9 depicts NO inactivation of sACE.

FIG. 10 depicts sACE on one immunocyte binding to sACE on another immunocyte so as to promote specific cell-cell interactions.

FIG. 11 a-g depicts the prompt response to angiotensin II receptor blockade in a 33 yr. Old which man with psoriasis.

FIG. 12 depicts the genetic pathway for initiation of benign prostatic heperplasia and prostate cancer.

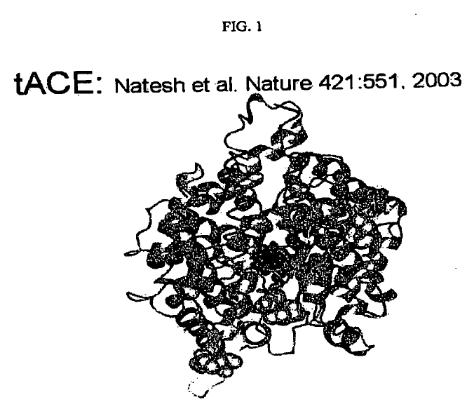

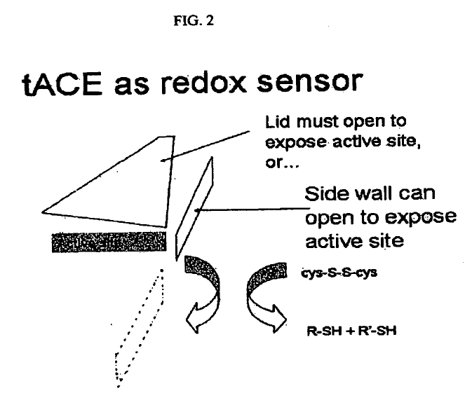

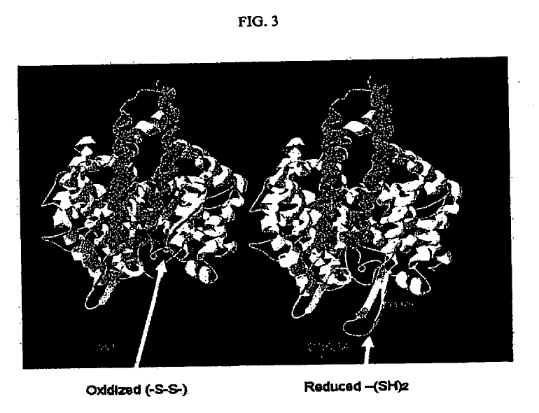

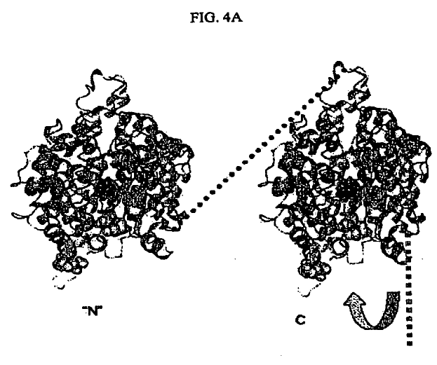

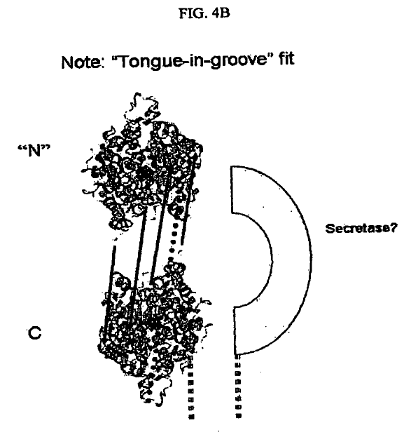

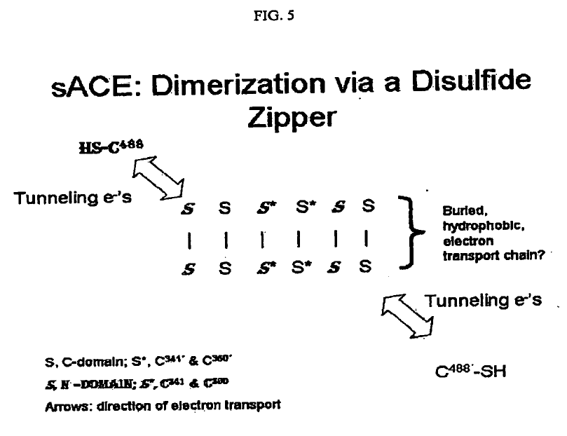

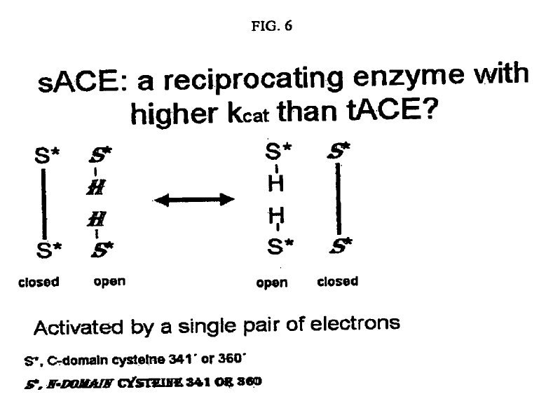

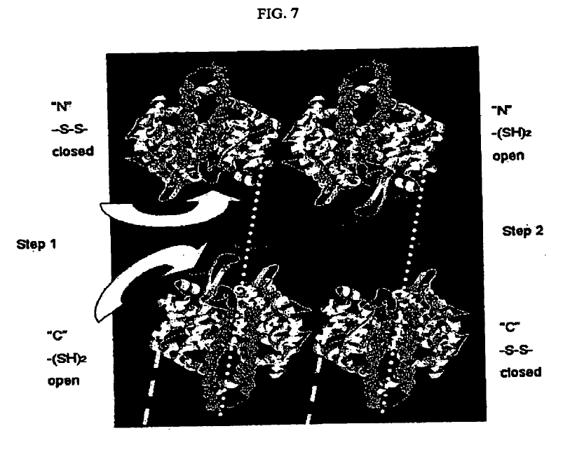

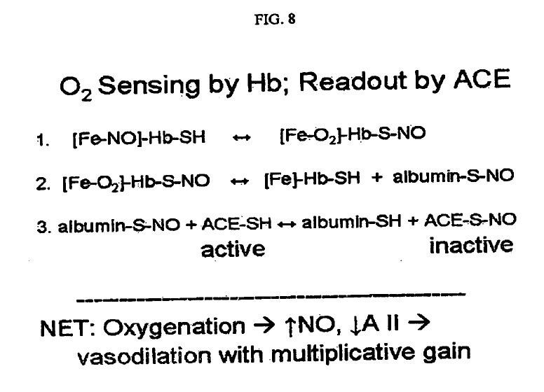

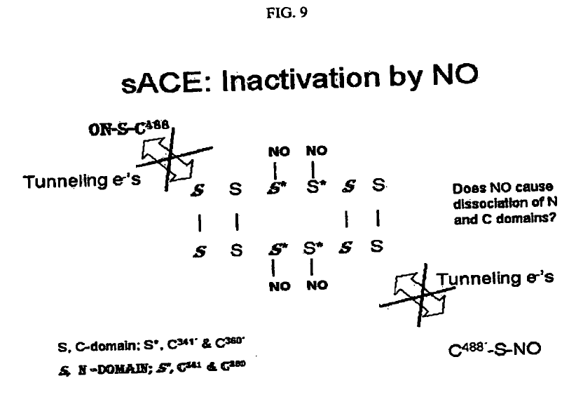

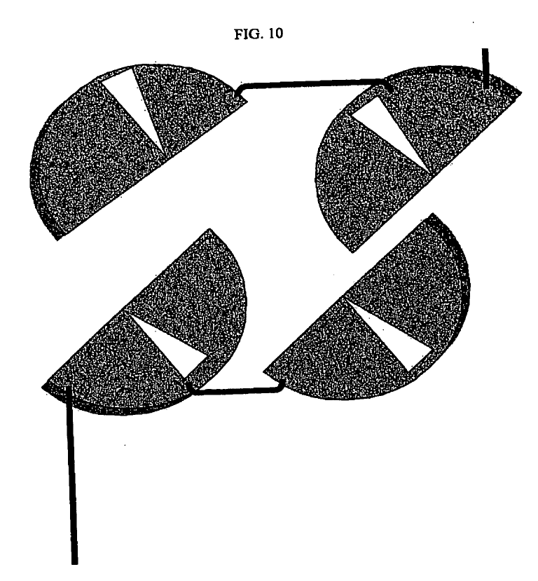

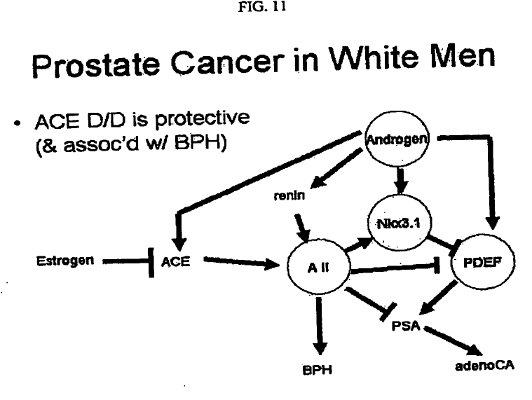

### DESCRIPTION

The present invention relates to methods for treating patients with various diseases, comprising adminisering an appropriate angiotensin II receptor inhibitor (ARB). Many ARBs are known in the art, any of which may be useful in the methods disclosed. Such currently known ARBs may be, for example, selected from the group consisting of irbesartan, candesartan, losartan, telmisartan, eprosartan and valsartan. It is also contemplated that other ARBs may be discovered, and it is expected that such newly discovered ARBs will be useful in the methods described herein.

The methods comprise treating patients with an angiotensin II receptor blocker. An angiotensin II receptor blocker is any molecule that reduces an effect of aniotensin II by hinders angiotensin II from binding to an angiotensin II receptor binding site.

The patient may be any mammal, including, for example laboratory animals such as mice, rats and guinea pigs; farm animals and domestic animals, including for example cows, sheep, pigs, goats, dogs and cats. The most preferred animals include primates such as monkeys, apes and humans. In some embodiments, the patient may display symptoms of, or be diagnosed with a disease or condition associated with overactivity of ACE.

The ARB may be administered at any time relative the onset of symptoms and diagnosis, and and by any acceptable means and via any acceptable route. ARBs may, for example, be administered intravenously, subcutaneously, interperitoneally, topically or orally. The ARB may be administered alone or in combination with other compounds.

As used herein, an “effective amount” of an ARB is any amount that inhibits angiotensin II from binding to an angiotensin II receptor. The effective amount of ARB can be routinely determined by a person of ordinary skill in the art. For example, the dosage may be about 0.1 mg/kg/day to upwards of 400 mg/kg/day. The preferred dosage is in the range of about 0.1 mg/kg/day to about 100 mg/kg/day. The ARB may be administered as a single daily dose, or several doses at various intervals.

The examples below are intended to be illustrative only, and the invention is not limited in any way to those embodiments discussed therein.

### Example 1

This example refers to the use of adequate tissue ACE inhibition or an AT1 receptor blocker (“ARB”) specifically for the autoimmune skin diseases psoriasis and alopecia greata, as well as related diseases: alopecia greata et totalis (AAT) and alopecia greata et universalis.

Autoimmune diseases in general reflect excessive activity of T cells. We have discovered a large number of autoimmune diseases associated with the ACE deletion/deletion genotype, which translates to overactivity of ACE (see U.S. Provisional Patent Application No. 60/512,458, filed Oct. 17, 2003, incorporated herein in its entirity). Both ACE and AT1 receptors are present on the surface (plasma) membrane of T cells and monocytes/macrophages, and the density of these 2 molecules increases upon activation immunologically. A logical treatment for autoimmune diseases is therefore to use effective ACE inhibition (as described in U.S. 60/512,458; U.S. patent application Ser. No. 10/213,330), or a selective angiotensin II receptor blocker (ARB) to treat the disease.

A. Psoriasis

In 2002, Dr. David W. Moskowitz began treating a 60 year-old white man with type II diabetes and severe psoriasis. The goal of the treatment was to achieve effective tissue ACE inhibition so as to prevent progression of the patient's diabetes. The patient took 200 mg/day quinapril, in 2 divided doses (120 mg at bedtime, 80 mg in the morning). This constituted a dose of −2 mg/kg/day (˜1 mg/lb/day).

Remarkably, the treatment also put the patient's psoriasis into remission, so that he no longer had to take daily methotrexate (75 milligrams), a chemotherapy drug.

B. Alopecia Areata

In this disease, a person's own T cells attack their hair follicles, causing patches of baldness. A 14 year old white girl whose alopecia greata could only be controlled by steroid injections into her scalp had hair loss every day for the past two months since her last scalp injections, despite using a potent steroid cream and an anti-viral cream on her scalp at night. A ball of hair the size of a half dollar would fill her shower drain every morning. Within 36 hours of starting an ARB (DIOVAN, or valsartan, 40 mg po qhs), her hair loss stopped. She stopped the steroid and anti-viral creams, and took the ARB, without any further hair loss. When she stopped the ARB after 1 week, her scalp hair loss resumed after 3-4 days.

Because many patients with an autoimmune disease such as alopecia greata, psoriasis, rheumatoid arthritis, multiple sclerosis, etc. are normotensive or even relatively hypotensive, an ACE inhibitor may not be the best choice for them. They might not be able to tolerate a sufficiently high dose of an ACE inhibitor, certainly any dose approaching 2 mg/kg/day. Instead, an AT1 receptor blocker appears to be effective at doses which cause minimal blood pressure (BP) reduction. For example, the patient with alopecia greata had her systolic BP reduced from 98 to 90 mm Hg by DIOVAN 40 mg po qhs.

Patients with even lower BP, e.g. younger children, could use a different AT1 receptor blocker since DIOVAN 40 mg is the smallest size pill, and it is a timed release tablet that does not permit easy breakage in half. Other more suitable ARBs include irbesartan (AVAPRO): its smallest size pill is 75 mg, but it can be easily broken in half to yield an even smaller dose, with less blood pressure lowering abililty. Similarly, ATACAND (candesartan) comes in a 4 mg pill, which can be broken in half; COZAAR (losartan) comes in a 25 mg pill, which can be broken in half; MICARDIS (telmisartan) comes in a scored 40 mg pill which can be broken in half; and TEVETEN (eprosartan) comes in a scored 400 mg pill which can be broken in half.

The above approach can also be used for any disease in which overactivity of the AT1 receptor is causative, and in which the patient is not hypertensive. Indeed, the relative effectiveness of DIOVAN, an AT1 receptor blocker in the case of alopecia greata suggests that it may sometimes be advantageous to use an AT1R blocker in autoimmunity or cancer. By not interfering with angiotensin II production, interruption of AT1 receptor signalling leads to induction of the ACE gene and even more angiotensin II production. If AT2 receptors are present, which are pro-apoptotic, then the presence of higher angiotensin II in the setting of working AT2 receptors but blocked AT1 receptors will result in amplification of the effectiveness of the AT1R blocker, out of proportion to its ability to lower blood pressure. In such a setting, an AT1R blocker may turn out to be more efficacious than the low dose of an ACE inhibitor which can be tolerated by the normotensive patient with an autoimmune disease or cancer.

### Example 2

AT1 Receptor Blockers (“sartans”) for Severe Acute Respiratory Syndrome (SARS)

The reason why the coronavirus kills in SARS is because of the exuberant host response, not because of tissue damage by the virus. Patients die of high fever and respiratory insufficiency. The lung interstitium is invaded by inflammatory cells, and alveoli fill with an inflammatory exudate. As a result, alveoli cease to become gas-exchanging units. Even in the absence of alveolar exudate, the distance between the alveolus containing oxygen-rich air and oxygen-transporting hemoglobin in the red cells of pulmonary capillaries widens because of the interstitial inflammation. Gas exchange becomes grossly impaired.

Similarly, coronavirus does not cause fever; the body's immune response does. Both interleukin-1 (IL-1) and tumor necrosis factor-α (TNF-α) are the pyrogens causing the high fever. But these interleukins are made by the host's T cells and antigen-presenting cells (APCs), including activated macrophages.

Decreasing the host's over-exuberant immune response to the coronavirus should reduce such symptoms.

It is our belief that angiotensin II is an as yet unrecognized major stimulator of the immune response. The rate-limiting step for its synthesis is the angiotensin I-converting enzyme (ACE). ACE is present on the plasma membrane of T cells and appears on the plasma membrane of antigen presenting cells (APCs) such as monocytes and macrophages once they have become activated.

Angiotensin II does a number of things. It probably stimulates the production of interferons by CD4+ T cells (helper T cells). CD4+ cells stimulate the activity of CD8+ cells (cytotoxic T lymphocytes, or CTLs). The primary job of CTLs is to kill virally infected epithelial cells.

Interferon-γ in particular is a major cytokine released by CD4+ helper T cells (T_{H}1 cells) in response to viral infection, and a major activator of CD8+ CTLs. Angiotensin II, operating through angiotensin II type 1 receptors (AT1Rs) appears to enhance the production of interferon-γ. Thus, angiotensin II operates as a very early amplifier of the host defense system against viral infection.

Angiotensin II also increases vascular permeability. It causes vasoconstriction of pulmonary arterioles in areas of inflammation, thus minimizing V/Q mismatch. Angiotensin II is probably the normal mechanism for controlling V/Q matching in the lung, in fact. When the pulmonary interstitium fills with inflammatory cells, or the alveolus itself fills with an inflammatory exudate and gas exchange becomes impaired, the arteriole leading to that alveolus undergoes vasoconstriction so blood no longer goes to the non-functioning alveolus. This physiological response is referred to as matching ventilation (V) with perfusion (Q). It likely occurs because ischemic tissue generates adenosine, which increases blood velocity, activating pulmonary endothelial ACE acting as a mechanosensor.

In the case of infection, ACE on T cells and activated macrophages (including resident alveolar macrophages) adds significantly to local angiotensin II production, further promoting vasoconstriction of the arterioles feeding the inflamed alveoli.

Two treatment possibilities appear promising. One is inhibition of ACE, but effective inhibition of tissue ACE requires a very high dose of ACE inhibitor, e.g. 2 mg/kg/d quinapril.

Another possibility is selective AT1R inhibition using an angiotensin II receptor blocker (“sartan”) such as valsartan (DIOVAN), irbesartan (AVAPRO), losartan (COZAAR), candesartan (ATACAND), telmisartan (MICARDIS), or eprosartan (TEVETEN). The lowest dosage should be used, and even these tablets should be split in half to minimize the danger of excessive lowering of blood pressure in volume-depleted acutely ill patients.

For example, an 80 mg DIOVAN capsule can be split in half, and 40 mg given once a day while the patient is in bed (e.g. at bedtime, or q am if the patient is already hospitalized). Irbesartan (AVAPRO) comes in 75 mg tablets which can be further split in half, and ˜37 mg given to the patient once a day.

The evidence for this approach is circumstantial at the moment. Until we have patient outcomes data for SARS, it will remain so. However, the alternative for SARS patients is to do nothing and run a 10% risk of acute mortality. The evidence consists of the following:
1. CD4+ T_{H}1 cells produce interferon-γ in alopecia greata; this disease, in its active form, can be shut down within 36 hours of starting valsartan 40 mg po qhs (n=1). In its more chronic form, it takes more than 5 days for valsartan to have an effect (n=1).
2. Infection by several common viruses, including hepatitis A and B, and HIV, are associated with overactivity of ACE, specifically the ACE deletion/deletion (D/D) genotype. So is infection with tuberculosis. So is progression of HIV to AIDS. [Ref. Moskowitz D W. Is ACE a ‘master’ disease gene? Diabetes Technology & Therapeutics 4(5): 683-711, 2002.] It appears that activation of T cells is an important step for viral replication. Angiotensin II is also important for many of the complications of HIV, such as HIV-associated nephropathy, and Kaposi's sarcoma. The latter is a tumor of hyperproliferating macrophages. Mesangial cell hyperplasia is the pathology of HIV-associated nephropathy; mesangial cells are essentially resident macrophages within the glomerulus.
3. Another autoimmune disease characterized by T cell autoimmunity, psoriasis, responded dramatically to high dose quinapril (2 mg/kg/day). A 62 yr old white man was able to stop his daily dose of 75 mg methotrexate after several months on high dose quinapril for diabetes and hypertension. Only residual psoriatic disease remains at the site of formerly exuberant disease.

### Example 3

I. Molecular Mechanism of Redox Sensing by ACE

Angiotensin I-converting enzyme (ACE) has a number of invariant cysteines, including a pair quite close to the “HEMGH” active site, as follows:

C127}

C135} 8 aa's “A”

C341}

C360} 19 aa's “B”

HEMGH 373-377 Zn⁺⁺-binding active site

C488 “A”

C531}

C549) 18 aa's “B”

This pattern is repeated in both the N- and C-terminal domains of the duplicated enzyme, somatic ACE (sACE).

It is believed that with the duplication, the molecule achieved the ability to be a redox sensor. When oxidized, in the presence of oxygen, the cysteines become linked to form cystines, i.e. Cys—SH+Cys—SH→Cys—S—S—Cys (cystine) (reaction 1)

Since the molecular structure of sACE has not yet been solved, it is clearly impossible to say which cysteines are cross-linked under oxiding conditions. However, it is attractive to speculate that the cysteines separated by 18-19 aa's line up with their counterparts and become cross-linked. In other words, the cysteines marked “B” line up and become cross-linked, and the cysteines marked “A” become cross-linked to their counterparts. In the latter case, C488 could pair with either C127 or C135. If the latter two cysteines are in an alpha helical domain, they would both be located on the same side of the helix, since an alpha helix recurs every 7 aa's.

This pairing and cross-linking could happen in the duplicated enzyme much more easily than in the single domain (testicular, or germinal) ACE, as follows:

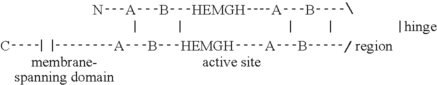

It is conceivable that cross-linking in such a fashion could limit access of substrate (angiotensin I, a decapeptide of molecular weight ˜1 Kd) to the active site of both the N and C terminal domains. This would be an additional “lock” on activity of ACE, on top of the ion- and flow-dependent “locks” previously described (in Diabetes Technology & Therapeutics 4(6):841, 2002).

Under reducing conditions, these intramolecular cystine bridges would be broken, and reaction (1) would be reversed. Chemical equilibrium would lie on the left side of the reaction, favoring Cys—SH rather than Cys—S—S—Cys. We postulate that both active sites would now be available for the 2^{nd} key to open them: an ionic key (chloride concentration above 50 mM) to open the C-terminal active site, and a mechanical key to open the N-terminal active site, as described in DT&T 4(6):841, 2002.

Reducing conditions include low oxygen tension, low pH, and high carbohydrate concentration.

II. Lung ACE: Low Oxygen Tension, High CO₂ Tension

This may explain how the lung matches ventilation (V) to perfusion (O). Normally, the lung rewards alveoli engaging in productive gas exchange with blood flow. Vessels supplying non-functional alveoli, in contrast, undergo vasoconstriction. The mechanism for matching Q to V has not been fully described, although the opening of K+ channels in vascular smooth muscle cells may be involved.

Here a much more straight-forward mechanism for matching V and Q is described. In pulmonary arterioles and capillaries, ACE is abundant on endothelial cell membranes, perhaps more abundant than in any other vascular bed. Pulmonary vessels are located in the interstitium, not more than a few cell lengths away from the gaseous phase in the alveoli. Oxygen diffuses from the alveolus to the blood vessels, is picked up by hemoglobin, and is transported, through the left side of the heart, to the rest of the body.

If the blood vessel has no oxygen to pick up because the alveolus nearby is non-functional, then the oxygen tension in the interstitium surrounding that alveolus will fall, and the carbon dioxide tension will rise, i.e. the pH will fall. Both lower oxygen tension and higher CO₂ tension constitute powerful reducing conditions.

Under such reducing conditions, it is postulated that the cystine bridges in ACE will be reduced to free cysteine sulfhydryl groups. The molecule will fall apart, exposing both active sites. The N-terminal active site will be activated by turbulent blood flow, and the C-terminal active site, fully active at the extracellular chloride concentration of 110 mM, will now be accessible to substrate as well. There will be a remarkable system gain with the removal of 2 locks, and ACE activity will increase in a multiplicative fashion. Local angiotensin II production will increase dramatically, and the vessel will constrict. This effect, if true, should be fully demonstrable using reducing agents in pulmonary arterial or arteriolar endothelial cells in vitro.

When gas exchange improves, oxygen tension in the interstitium and at the plasma membrane of the endothelial cell increases, the free cysteine sulfhlydryl groups become oxidized to cystine once again, the ACE molecule gets “locked” up, and inactivated. As a result, angiotensin II production drops dramatically, and the balance between vasoconstriction (mediated by angiotensin II and its induced downstream partners, endothelin, etc.) and vasodilatation (mediated by nitric oxide, NO) shifts in favor of the vasodilators. The pulmonary capillary opens up, and blood flow now services that alveolus again.

If alveolar gas exchange is impaired for a prolonged period, long-term effects of angiotensin II include the following: hyperplasia of vascular smooth muscle cells leading to pulmonary hypertension; elaboration of TGF-beta and proliferation of interstitial fibroblasts leading to pulmonary fibrosis.

Thus, effective tissue ACE inhibition and/or angiotensin II type 1 receptor antagonism (by “sartan” drugs, or ARBs) is expected to be useful for any pulmonary disease in which gas exchange is impaired, including but not limited to emphysema, bronchiolitis obliterans, acute respiratory distress syndrome, severe acute respiratory syndrome (SARS), and all other forms of pulmonary fibrosis, as well as lung cancers which probably arise by hypoxemia-induced production of angiotensin II, which is a growth factor.

Note: Hyperoxia and Pulmonary Fibrosis

This molecular mechanism does not readily explain how hyperoxia, as in prolonged mechanical ventilation using an F_{I}O2≧0.5, could result in pulmonary fibrosis in adults and newborns. Indeed, ACE should be “locked up” by hyperoxia. However, a high F_{I}O2 is only used clinically in situations of impaired gas exchange. In the regions of impaired gas exchange, oxygen tension can be quite low, and cystine groups within the ACE molecule will actually be reduced in exactly the same way as described above. Hyperoxia creates oxygen free radicals, which react with nitric oxide to produce peroxynitrate, ONOO⁻, as recently described by Tarpey et al. from UAB, Alabama. Thus, hyperoxia displaces the balance between angiotensin II (being generated in regions with poor gas exchange) and NO even more to the side of angiotensin II by depleting NO.

III. Arterioles and Capillaries Outside the Lung: Sepsis, DIC, Malignant Hypertension, Sickle Cell Disease

This same mechanism (low oxygen tension, low pH) also exists in other tissue vascular beds, such as liver and muscle, during conditions of hypoperfusion, e.g. in cardiogenic or hypovolemic or septic shock, sickle cell disease, as well as in complications of excessive ACE activation, as in malignant hypertension or DIC (disseminated intravascular coagulation). DIC is particularly instructive, since it is a currently untreatable exit for patients with irreversible sepsis or shock, or untreated malignant hypertension.

The pathophysiology of DIC is hypercoagulation. This could arise by a profound imbalance between angiotensin II, which is profoundly pro-thrombotic, and NO, which is anti-thrombotic. In addition, endothelial cell ischemia and apoptosis will increase coagulation on the vascular wall.

Sickle cell “crisis” is similar to DIC in occurring within the vasculature, and involving a vicious cycle of vasoconstriction, hypoperfusion, hypoxemia, acidemia, and worse sickling of red cells. ACE inhibition or the use of angiotensin II receptor blockers (ARBs) or both, either orally or intravenously (the latter for patients who are vomiting and can't keep down pills) is proposed as a prophylaxis against sickle cell crisis, as well as a treatment for it.

IV. Hyperglycemia: Diabetes and Activation of ACE

Glucose, unlike fructose, has a C1 aldehyde which can be oxidized to a carboxyl group. Fructose has a C2 ketone which cannot be further oxidized. Glucose, but not fructose, is therefore a powerful reducing agent. The hyperglycemia of diabetes, in addition to raising plasma osmolality slightly (see DT&T 4(6), 2002 and 5(2), 2003), also provides a high concentration of reducing agents to ACE on endothelial cell walls. The result is reduction of cystines to free cysteines, and activation of ACE as described above. This effect is probably more important than the osmotic effect of hyperglycemia to increase mechanical shear stress on the N-terminal active site, hypothesized in DT&T 4(6), 2002.

V. Link between Diabetes and Gout

A number of diseases associated with excess angiotensin II are linked to gout, such as hypertension, coronary artery disease, and NIDDM. It appears that angiotensin II stimulates production of xanthine oxidase (XO) by endothelial cells in an autocrine/paracrine fashion. XO is expressed on the plasma membrane of endothelial cells in the same location as ACE. XO converts xanthine and hypoxanthine to uric acid. Uric acid production is linked to gout, since gouty crystals are pure uric acid.

XO can create uric acid through electron transfer to its Molybdenum VI center, and thence to an iron-sulfur protein, and thence to a flavin moiety. But XO can also create free oxygen radicals through its flavin center alone. These free oxygen radicals deplete NO by creating ONOO—, as mentioned above (Tarpey et al.). Thus, synthesis of XO by angiotensin II may be a way for the vasoconstrictor, prothrombotic, profibrotic, proapoptotic pathway to gain the upper hand over the vasodilatory, antithrombotic, antiproliferative and antifibrotic pathway of NO.

Thus, gout should be amenable to treatment with inhibitors of All synthesis (ACEI's) or action at the type 1 receptor (ARBs).

VI. Renal Hypoperfusion and Acute Oliguric Renal Failure

Besides adenosine, angiotensin II may be involved in ARF due to hypoperfusion, either due to hypovolemia or pump failure. By creating a hypoxic environment within the kidney, and thus reducing conditions, ACE in the proximal tubular brush border membrane may be activated directly, adding to vasoconstriction by adenosine. Thus, ACE inhibition and/or ARBs may prevent against pre-renal ARF.

Note: Why do oxidizing agents induce chronic renal failure?

Since ACE activation is the major mechanism for chronic renal failure, and oxidation is postulated here to “lock up” ACE, why do oxidizing agents produce a situation of ACE overactivity? This paradox resembles that of pulmonary fibrosis due to hyperoxia, and may have a similar explanation. By increasing the concentration of oxygen free radicals, oxidizing agents may deplete NO in renal vessels. Although ACE is largely inactive because of these oxidizing conditions, there will be basal activity of ACE from the C-terminal domain due to chloride activation, and the N-terminal domain through mechanical activation. The effect of this small amount of angiotensin II will be amplified paradoxically by oxidizing agents which deplete NO. Renal vasoconstriction will result, further increasing mechanical shear forces on ACE. Vasoconstriction will also result in tissue hypoxia, promoting reduction of cystine bridge(s) on ACE, and further activation.

VII. Role of ACE in Inflammation

ACE is present on T cells and activated macrophages. Bacterial infection produces anaerobic conditions. The bacteria either consume oxygen themselves, or are anaerobes. Along with tissue hypoxia, the pH of bacterial infections, except for urease producing bacteria in the urinary tract, is always low. Under these reducing conditions, ACE should become activated.

Angiotensin II is a potent cytokine. It stimulates macrophages to make macrophage migration inhibition factor (MIF) and TNF-alpha, among other cytokines.

Angiotensin II stimulates T cells to secrete interferon-gamma. These cytokines amplify the response of the immune system to the invading microbes. Angiotensin II also stimulates apoptosis of virally infected pulmonary epithelial cells, helping to decrease spread of virus to nearby cells.

Once activated, macrophages produce oxygen free radicals, which they use to kill bacteria and viruses. (So do polymorphonuclear leucocytes, or PMNs, but PMNs lack ACE on their membrane). Production of oxygen free radicals by activated macrophages is predicted by the hypothesis advanced here to turn off macrophage ACE in an autocrine fashion, and endothelial cell ACE in a paracrine fashion. This should result in tipping the balance seen by vascular smooth muscle cells away from angiotensin II and towards NO, with the result that vasodilatation occurs.

This is teleologically advantageous, since blood flow is all that is needed to clear most infections.

### Example 4

Torticollis appears to involve excessive sympathetic nervous discharge from cervical nerves to neck muscles. Imbalance in nerve firing results in a twisting of the neck (torticollis' meaning in Latin) to one side.

Nerve firing is excessive often because of inflammation. Other associations are with trauma, and genetics (e.g. family history of epilepsy). The disease's predominance among women between 30 and 60 suggests an autoimmune origin for the most common form of torticollis.

Here, the use of an ARB is suggested, especially for patients with torticollis and normal or low blood pressure. An ideal ARB might be eprosartan, since it blocks angiotensin II type 1 receptors on both sympathetic pre-synaptic nerve terminals and post-synaptic muscle cells. A starting dose for a patient with low blood pressure (below 100 mm Hg systolic) might be 100-150 mg po qhs (eprosartan is available commercially as 400 and 600 mg tablets; the starting dose would be ¼ of a tablet; pill splitters are available in most drug stores or grocery supermarkets).

Neurodegenerative diseases like the spinal muscular atrophy syndromes (types I and II), and amyotrophic lateral sclerosis (ALS, “Lou Gehrig's disease”) appear to involve the final common pathway of oxidative damage and apoptosis. The molecular defect in SMA appears to be a defect in processing messenger RNA due to a defective protein component of the spliceosome; the defect in ALS is lack of superoxide dismutase (SOD).

Angiotensin II contributes mightily to oxidative stress, through activation of mitochondrial NAD(P)H oxidase. Angiotensin II also contributes to apoptosis in various cell types, including renal tubular epithelial and pulmonary epithelial cells, and perhaps neurons.

One would reasonably expect the safe and well-tolerated class of ARBs to be effective in treating neurodegenerative diseases such as SMA and ALS. Again, because of blockade of pre-synaptic nerve terminals, eprosartan may be one of the more effective ARBs to use.

### Example 5

This example describes treatment for age-related macular degeneration (ARMD).

The macula is a part of the retina furthest removed from a blood supply. As a result, it is chronically hypoxic. With age, it undergoes neovascularization. Antioxidants have been useful in treating it (ref.:Minn Med. 2003 April;86(4):40-6. New treatments for age-related macular degeneration. Miftra R A. VitreoRetinal Surgery, P.A., Edina, USA.).

Since we posit that ACE is activated by hypoxia (reducing conditions), it is reasonable to expect that production of angiotensin II should produce both neovascularization (All is a potent angiogenesis factor) as well as oxidizing species (All stimulates transcription of mitochondrial NAD(P)H oxidase, a rate-limiting step for oxygen free radical production).

Inhibition of angiotensin II production with an ACE inhibitor, preferably one which can penetrate into the eye (e.g. a topical ACE or an oral hydrophobic ACE inhibitor), or an angiotensin II receptor blocker, should therefore be useful in arresting and perhaps even reversing the progression of ARMD.

This approach would also be expected to work for other retinal diseases characterized by neuronal loss and/or neovascularization, such as retinitis pigmentosa, diabetic retinopathy, etc.

### Example 6

Respiratory syncytial virus (RSV) is a cause of significant morbidity and mortality among infants and the elderly and immunocompromised in the US and elsewhere. RSV pneumonia can lead to chronic sequelae in children such as bronchiolitis obliterans and asthma, which are characterized by exaggerated T(helper)2 activation, and secretion of cytokines such as interferon-gamma (1-3). T cells, when activated, express angiotensin I-converting enzyme (ACE) on their surface membrane. ACE is the rate-limiting step for production of angiotensin II, which activates cells of the innate immune response such as macrophages and T lymphocytes through AT1 receptors.

RSV pneumonia is characterized by a T cell lung infiltrate and apoptosis of virally infected lung epithelial cells (4). Angiotensin II promotes pulmonary epithelial cell apoptosis.

An exaggerated pulmonary immune response suggests that immunosuppression might be useful, e.g. systemic corticosteroids. The problem with immunosuppressing a patient with suspected RSV pneumonia is that clinicians are reluctant to use strong, systemic immunosuppression in a situation in which the patient's decline may be due to an unrecognized or inadequately treated micro-organism. Strong immunosuppression under these circumstances could lead to rapid death from disseminated infection. As a result, systemic corticosteroids are reserved for severe cases of RSV, and are of limited utility (5).

A method of mild immunosuppression is therefore required for safe, efficient treatment of patients with RSV pneumonitis to hasten resolution of airway disease, and to try to prevent progression to bronchiolitis and asthma.

Both macrophages and T cells express angiotensin I converting enzyme (ACE) on their surface membrane when activated. The product of ACE, angiotensin II, appears to act in an autocrine and paracrine fashion to activate macrophages and T cells. In other words, angiotensin II is an under-appreciated cytokine. We have found that an ARB can halt active alopecia greata, which is mediated by T lymphocytes, and an ACE inhibitor can decrease the intensity of psoriasis. Both are classic autoimmune diseases.

It is therefore reasonable to expect treatment by the use of an angiotensin II receptor blocker (ARB) such as irbesartan 37.5 or 75 mg once or twice a day to infants with suspected RSV pneumonitis to be successful. Higher doses, or a hydrophobic ACE inhibitor such as quinapril, could be used for people with hypertension. At this point there's no reason to think this wouldn't be a class effect, although it is possible that some ARBs may turn out to be superior to others in their effectiveness. For patients with significant hypertension, treatment with a hydrophobic ACE inhibitor such as ramipril, quinapril, or trandolapril ought to be effective, although ACE inhibitors produce a mild cough in up to 10% of patients.

### REFERENCES

1: Pediatr Infect Dis J. 2003 February;22(2 Suppl):S76-82. Respiratory syncytial virus bronchiolitis and the pathogenesis of childhood asthma. Martinez FD. Arizona Respiratory Center, University of Arizona College of Medicine, PO Box 245030, 1501 N. Campbell Avenue, Room 2349, Tucson, Ariz. 85764, USA. fernando@resp-sci.arizona.edu
2: Acta Paediatr Suppl. 2002;91 (437):6-11. Origins of reactive airways disease in early life: do viral infections play a role? Piedimonte G. Batchelor Children's Research Institute, Pediatric Pulmonology and Cystic Fibrosis Center, University of Miami School of Medicine, 1580 NW 10th Ave. (D-820), Miami, Fla. 33136, USA. gpiedimo@med.miami.edu PMID: 12200895
3: Immunopharmacology. 2000 Jul. 25;48(3):237-47. Immune-mediated disease pathogenesis in respiratory syncytial virus infection. Graham B S, Johnson T R, Peebles R S. Department of Medicine, A-4103 MCN, Vanderbilt University School of Medicine, 1161 21st Avenue South, Nashville, Tenn. 37232-2582, USA. barney.graham@mcmail.vandrbilt.edu PMID: 10960663
4: Am J Pathol. 2002 December; 161(6):2195-207. Replication and clearance of respiratory syncytial virus: apoptosis is an important pathway of virus clearance after experimental infection with bovine respiratory syncytial virus. Viuff B, Tjornehoj K, Larsen L E, Rontved C M, Uttenthal A, Ronsholt L, Alexandersen S. Department of Pharmacology and Pathobiology, The Royal Veterinary and Agricultural University, Ridebanevej 3, DK-1870 Frederiksberg C, Denmark. bmv@kyl.dk PMID: 12466134
5: AACN Clin Issues. 2002 August;13(3):421-30. Management of respiratory syncytial virus with lower respiratory tract infection in infants and children. Garzon L S, Wiles L. School of Nursing, Old Dominion University, Norfolk, Va. 3529, USA. Igarzon@odu.edu PMID: 12151995

### Example 7

Use of Angiotensin II Blockade Against All Viral Diseases

Viruses, especially those which rarely infect humans, can be quite lethal. An example is the novel influenza A strain which caused the 1918 flu pandemic. In large part, this appears to be due to lack of a sufficiently developed suppressor response to the virus, either in the form of passive opsonization by pre-existing antibodies or memory suppressor cell (T_{H}2) responses.

Respiratory viruses such as influenza (1), hantavirus (2), RSV (3), SARS (4), etc. cause death due to an acute respiratory distress-like syndrome (ARDS) caused by an overly exuberant innate immune response to the novel virus. Hemorrhagic viruses like Ebola virus (5) cause DIC via a similar mechanism: an overly exuberant immune response to a novel virus. Smallpox causes a systemic disease with a 30% mortality which clearly involves massive activation of the innate immune response. The same is true for viruses which cause localized CNS damage, such as West Nile virus, St. Louis encephalitis, Eastern Equine encephalitis virus, and polio. The host's immune response, not viral overgrowth, determines whether the host lives or dies.

Angiotensin II is a critical cytokine for the innate immune response, since ACE, the enzyme which produces angiotensin II, is expressed on activated macrophages and T cells. All immune cells, including macrophages, T and B cells, and neutrophils, contain AT1 receptors. Blocking angiotensin II production with a suitable ACE inhibitor at a suitable dose, or action of angiotensin II at AT1 receptors with an angiotensin II receptor blocker (ARB) would be expected to decrease mortality from all viral infections.

Table 2. Response of eight patients with West Nile virus encephalitis to angiotensin II receptor blockade. Seven patients showed an unexpectedly prompt response, for an 88% treatment success rate.
1. A 50 yr old white man with past history of tremors was admitted with fever, stiff neck, severe confusion, and gross tremors to a hospital in Omaha, Nebr. one night. His serum was positive for IgM reactive to West Nile virus. He was begun on olmesartan 20 mg daily. On the morning after admission, his tremors had lessened dramatically, and he was able to concentrate normally. He was discharged home on olmesartan 20 mg daily.
2. A 50 yr old white woman with hypertension treated with a beta-blocker/thiazide combination drug, was admitted to a hospital in Omaha, Nebr. with right leg paralysis and paresthesias and weakness in her left leg. Her serum was positive for IgM reactive to West Nile virus. She was begun on losartan 50 mg daily. The paresthesias and weakness in her left leg stopped within 1-2 days of starting losartan, but there was no change in her right leg paralysis.
3. A 75 yr old white man with a history of seizures was admitted one night to a hospital in Omaha, Nebr. with a fever and a grand mal seizure. He had been taking olmesartan 20 mg daily for mild hypertension. His serum was positive for IgM reactive to West Nile virus. He was given losartan 50 mg twice during the first 24 hr after admission. His usual dose of olmesartan 20 mg daily was resumed thereafter. After his presenting seizure, he had no more seizures and had a full recovery within 12 hr. He was able to use his son's lap-top computer the morning after admission.
4. A 50 yr old white man was admitted with fever, stiff neck, and severe confusion to a hospital in Omaha, Nebr. His serum was positive for IgM antibodies reactive to West Nile virus. He was begun on losartan 50 mg daily. His meningoencephalitis disappeared within 48 hr.
5. A 73 yr old white man was admitted with fever, stiff neck, and severe confusion to a hospital in Omaha, Nebr. His serum was positive for IgM antibodies reactive to West Nile virus. He was begun on losartan 50 mg daily. His meningoencephalitis disappeared within 48 hr.
6. A 17 yr old white woman was admitted with fever, stiff neck, and severe confusion to a hospital in Pueblo, CO. Her serum was positive for IgM antibodies reactive to West Nile virus. She was begun on losartan 50 mg daily. Her meningoencephalitis disappeared within 24 hr.
7. An 80 yr old white man who had meningoencephalitis with positive IgM antibodies for West Nile virus in early August, 2003 was seen at a hospital in Pueblo, CO because of residual weakness and fatigue one month later. He was begun on losartan 50 mg daily, with disappearance of his weakness and fatigue within 24 hr.
8. A 40 yr old white woman with chronic lymphocytic leukemia was admitted with fever and obtundation to a hospital in Pueblo, CO. Her serum was positive by EIA for West Nile virus antibodies. She was given intravenous enalapril on the night of admission followed by losartan 50 mg daily, but remained in coma. Head CT and MRI scans were negative.

### REFERENCES

1. Rimmelzwaan G F, Kuiken T, van Amerongen G, Bestebroer T M, Fouchier R A, Osterhaus A D. A primate model to study the pathogenesis of influenza A (H₅N1) virus infection. Avian Dis. 2003;47(3 Suppl):931-3. PMID: 14575089
2. Schussler J M, Jordan H, Stokoe C, Feliciano N, Sutker W, Orr D. Hantavirus pulmonary syndrome. South Med J. 1999 February;92(2):233-5. PMID: 10071675
3. Kilani R A. Respiratory syncytial virus (RSV) outbreak in the NICU: description of eight cases. J Trop Pediatr. 2002 April;48(2):118-22. PMID: 12022427
4. Rubenfeld G D. Is SARS just ARDS? JAMA. 2003 Jul. 16;290(3):397-9.
5. Geisbert T W, Young H A, Jahrling P B, Davis K J, Larsen T, Kagan E, Hensley L E. Pathogenesis of Ebola Hemorrhagic Fever in Primate Models: Evidence that Hemorrhage Is Not a Direct Effect of Virus-Induced Cytolysis of Endothelial Cells. Am J Pathol. 2003 December; 163(6):2371-82.

### Example 8

Vitiligo is an autoimmune disease characterized by loss of skin pigmentation. It usually begins around age 20 (2) and affects patches of skin, but can progress to involve the entire body. Vitiligo appears to be due to an autoimmune attack by a person's T lymphocytes against the skin cells that produce pigment, the melanocytes. Although not life-threatening, the disease can be disfiguring. Vitiligo affects perhaps 500,000 people in the United States, and many more worldwide. Current treatment is expensive and not available everywhere. It is reasonable to expect that treatment of vitiligo with an effective dose of ACE inhibitor or ARB would reduce symptoms of this disease.

### REFS

(1) Mandelcorn-Monson R L, Shear N H, Yau E, Sambhara S, Barber B H, Spaner D, DeBenedette M A. Cytotoxic T lymphocyte reactivity to gp100, MelanA/MART-1, and tyrosinase, in HLA-A2-positive vitiligo patients. J Invest Dermatol. 2003 September;121(3):550-6. PMID: 12925214
(2) Das S K, Majumder P P, Chakraborty R, Majumdar T K, Haldar B.Genet Epidemiol. 1985; 2(1):71-8. Studies on vitiligo. I. Epidemiological profile in Calcutta, India. PMID: 4054593

### Example 9

It has recently been found (1) that many neuropsychiatric diseases, including bipolar affective disorder (manic-depression), schizophrenia, and seizure disorder, are associated with overactivity of the angiotensin 1-converting enzyme (which is abbreviated as ACE). Although we have no genetic evidence for autism and attention deficit and hyperactivity disorder (ADHD), it is likely that the cognitive and behavioral abnormalities seen in autism and ADHD may be the result of an imbalance of neurotransmitter levels in the brain, especially catecholamine levels, and dopamine in particular (2,3).

Angiotensin II, the product of ACE, is an under-appreciated neurotransmitter. It acts synergistically with catecholamines, promoting dopamine and norepinephrine release and reuptake in the peripheral (and presumably the central) nervous system. Both dopamine and angiotensin II stimulate thirst and the drinking of water, for example. Angiotensin II does this via the type 1 receptor (AT1R) (4).

The practical value of this approach is that ACE inhibitors and angiotensin II type 1 receptor blockers (“ARB's”) are safe drugs, even for young children. They have been in very widespread clinical use for over two decades. Literally hundreds of millions of people have used them. The only behavioral symptom noted is an elevation in mood in 20% of adult patients, consistent with the association we've seen between depression and overactivity of ACE. They can be taken by mouth, and are relatively inexpensive.

This trial therefore would consist of using very safe drugs to try to treat a difficult disease. It is especially useful for children who are not doing well with conventional drugs, e.g. Ritalin for ADHD. The clinical hypothesis is that blockade of angiotensin II action would improve behavior and cognition in children or adults with ADHD.

For the extremely rare child, or the more common adult, with hypertension, a hydrophobic ACE inhibitor would be prescribed. But most children have a low blood pressure. For them, the smallest dose of an ARB already approved for use in children, such as irbesartan (AVAPRO), could be used safely—with even fewer side effects than an ACE inhibitor.

For example, AVAPRO could be used in its lowest dose (a 75 mg pill), or the 75 mg pill could be further divided in half or thirds, in an attempt to avoid lowering the child's blood pressure at all.

The study would involve treating children with autism or ADHD in an open-label fashion with AVAPRO once a day (at bedtime), and following the children's behavior for several months (the longer, the better). Practically speaking, 6 months should be enough to see if there's any difference. AVAPRO would be prescribed by the child's pediatrician if s/he agreed to participate in the trial.

### REFERENCES

1. Moskowitz D W. Is angiotensin I-converting enzyme a “master” disease gene? Diabetes Technol Ther. 4(5):683-711, 2002.
2. Nieoullon A. Dopamine and the regulation of cognition and attention. Prog Neurobiol. 67(1):53-83, 2002.
3. Galili-Weisstub E, Segman R H. Attention deficit and hyperactivity disorder: review of genetic association studies. Isr J Psychiatry Relat Sci. 40(1):57-66, 2003.
4. Bagi E E, Fekete E, Lenard L. Angiotensin II and III microinjections into the zona incerta influence drinking behavior. Brain Res. 977(2):199-208, 2003.

### Example 10

Symptoms of the common head cold consist of sore throat, runny nose, sinus drainage, and occasionally fever, muscle aches, weakness, and fatigue. The common cold causes billions of dollars of lost work. It is caused by a number of viruses well known to our species, including human coronaviruses. SARS is caused by a coronavirus which is new to humans.

Genomic epidemiologic evidence suggests that autoimmune diseases such as allergic sinusitis are associated with overactivity of angiotensin I-converting enzyme, or ACE. Clinical evidence suggests that blocking the major product of ACE, which is a small eight amino acid hormone named angiotensin II, can decrease inflammation in a number of diseases, including viral disease. These diseases include alopecia, psoriasis, fibromyalgia and chronic fatigue syndrome, and West Nile virus encephalitis.

Using angiotensin II blockers to block the symptoms of a viral illness such as the common cold has enormous clinical appeal, since nobody taking an angiotensin II blocker or ACE inhibitor in the 25 years of their global use has ever appeared to be immunocompromised. The same cannot be said for any other immunomodulator in clinical use, such as steroids, which cause a number of problems when used chronically. Not so with angiotensin II blockers.

A preferred treatment would be to begin administering an ARB or ACE inhibitor when a patient feels a cold coming on, not after the patient feels so miserable that s/he has to stay home from school or work.

### Example 11

IDDM (“juvenile-onset diabetes mellitus) affects 5 million people in the U.S., and millions more in Europe and around the world. The disease-predisposition genes for IDDM have recently been found. To date, there are approximately 15 loci known, to varying degrees of specificity. This application describes a treatment to prevent IDDM in genetically susceptible children.

How to Establish Genetic Susceptibility

Patients can be genotyped for any of the IDDM-associated genes discovered so far. An alternative, for children with a history of IDDM already in their family, is to assume that they have a sufficiently elevated risk of contracting the disease that prophylaxis is warranted. In other words, if an older sibling contracts IDDM, then all younger siblings should be placed on prophylaxis. If a parent or cousin has IDDM, prophylaxis is also indicated.

Prophylaxis

Prophylactic treatment consists of an angiotensin II receptor blocker (ARB) begun at an early age, perhaps as early as 2 years of age, but no later than 9 years of age. The ARB should be approved for use in children, such as AVAPRO^{R} (irbesartan). The dose used should not result in any appreciable decrease in systemic blood pressure. For a 2 year old weighing 25 pounds, a 75 mg AVAPRO pill cut into quarters could be used as follows: one-quarter pill (18.75 mg or thereabouts) at bedtime daily. For a 9 year old weighing 70 pounds, a 37.5 mg dose of AVAPRO may be suitable.

Treatment would proceed for the life of the patient, since there are many additional autoimmune diseases which patients with IDDM are at higher risk for, relative to the general population. These include Hashimoto's autoimmune thyroiditis, alopecia, psoriasis, and the like.

### Example 12

Ricin poisoning results from the inhalation, ingestion, or subcutaneous injection of very small quantities of ricin, a natural product of the castor bean. Less than 1 mg is sufficient to kill an average adult. Ricin is a 65 kD heterodimeric glycoprotein consisting of two chains, the A and the B chain, covalently linked by a disulfide (cystine) bond. Ricin is glycosylated, containing some 15 moles of mannose and 8 moles of N-acetylglucosamine per mole of ricin. The B chain binds to galactose-containing glycolipids and glycoproteins on the cell surface, and induces endocytosis of the holoprotein. Once inside the cell, ricin undergoes reverse transport from the Golgi to the ER. In the ER, the A chain unfolds and is translocated into the cytoplasm for degradation, as part of the ER-assisted degradation (ERAD) pathway. The A chains that elude proteasomal degradation in the cytoplasm bind to 28S rRNA on the large subunit of the ribosome, and depurinate it, removing adenine bases specifically. The net effect is that the large ribosomal subunit loses its tertiary structure, and protein synthesis (elongation) is halted. This is sufficient to induce apoptosis of the cell.

Ricin is extremely potent; it is estimated that a single molecule can kill a cell.

Ricin at smaller doses has a laxative effect, and is the active ingredient of castor oil, long used as a laxative.

Death from ricin requires a lag period of 12-24 hours. Ricin affects the reticuloendothelial system primarily. Inhaled ricin results in apoptosis of alveolar macrophages. Ingested ricin crosses the gut and enters the liver via the portal vein. Once in the hepatic sinuosoid, ricin preferentially affects Kuppfer cells, which are macrophages within the liver sinusoids. Macrophage apoptosis leads to massive release of TNF-alpha, with vomiting, fever, and an endotoxic shock like picture. Terminally, the picture can resemble DIC, with bleeding.

TNF-alpha secretion by macrophages occurs within 6 hr of exposure to a number of toxic insults besides ricin, including viruses, UV light, and LPS (lipopolysaccharide derived from Gram negative bacterial cell walls). PKC, especially PKC-epsilon (PMID 8490103), is an early step in this pathway (PMID 12867362). PKC alpha and xi are also abundant in macrophages, and may participate in signaling. Downstream signaling is performed by the MAPK's: JNK which stimulates transcription of TNF-alpha, as well as ERK and p38 which stabilize TNF-alpha mRNA. ERK is important in the increase in TNF-alpha message in alveolar macrophages but not peritoneal macrophages (PMID 10857863).

Glucocorticoids inhibit JNK and decrease TNF-alpha secretion. Aspirin (acetylsalicylic acid) also inhibits TNF-alpha secretion.

TNF-alpha acts through the two TNF receptors, p55 and p75, to release ceramide and sphingosine, which promote cell death by apoptosis (PMID 12688321).

About 12 hr after exposure to toxic insults, iNOS is induced and generates high levels of NO. NO induces macrophage apoptosis via p53 and Bax. Aspirin inhibits NO production as well. NO is in part responsible for the hypotension seen as part of endotoxic shock.

Vomitoxin, a fungal product, acts similarly to stimulate MAPK's within macrophages to transcribe TNF-alpha, a function of p38 and ERK, as well as stabilize TNF-alpha mRNA (a function of p38).

TNF-alpha has numerous effects. One is to increase the level of concentrative nucleoside transporters (cnt 1 and 2) in macrophages (PMID 11346649). This may have relevance to activation of sporozoites by UDP observed by Maria Mota, Ana Rodriguez, and colleagues (PMID12379848; PMID 11835279). Perhaps TNF-alpha stimulated Kuppfer cells have higher UDP levels, leading to greater activation of sporozoites and hence greater infectivity of Plasmodium species. Individuals whose Kuppfer cells were capable of synthesizing high amounts of TNF-alpha would be the best hosts for malaria.

They would also succomb the fastest to ricin.

The ACE deletion/deletion (D/D) genotype is associated with a number of macrophage-dependent diseases, including TB and HIV infectivity (see table 17 of PMID 12458570). ACE is expressed on endothelial cells lining the liver sinusoid, as well as on macrophages (including Kuppfer cells, presumably) when they get activated. Patients with the D/D genotype express more ACE than those with the I/D or I/I genotype (refs. summarized in PMID 12396747). Macrophages possess AT1 receptors, which are activating (PMID 14716205; 12090726). They may also possess AT2 receptors (PMID 10404956), which are usually pro-apoptotic (PMID 12824823; 12797627).

We hypothesize that hepatic Kuppfer cells are normally activated by angiotensin II synthesized by ACE on nearby endothelial cells. ACE may be a mechanosensor and redox sensor, and is the rate-limiting step for synthesis of angiotensin II. Kuppfer cells, bathed in angiotensin II, will have a higher than zero activation of PKC via AT1 receptors.

It is reasonable to assume that PKC activation must reach a certain, fairly high threshold before downstream signaling via MAPK's lead to increased TNF-alpha secretion. Toxic insults such as viruses, UV light, ricin, malarial sporozoites, and LPS, will stimulate PKC further.

With reduced baseline levels of PKC stimulation, relative protection against viruses, UV light, ricin, malarial sporozoites, and LPS should be afforded.

This can be done with AT1 receptor blockers (ARBs) or ACE inhibitors. Thus, additional uses of ARBs and ACE inhibitors include: protection against hepato-tropic viruses, such as hepatitis A, B, and C (see Table 12 of PMID 12458570); protection against malaria by Plasmodium falciparum and related species; and protection against ricin poisoning.

In the case of ricin poisoning, a dose of an ARB, e.g. losartan 12.5-50 mg, would be given by mouth immediately, with subsequent doses given every 12 hours as allowed by the patient's blood pressure. An equivalent amount of irbesartan (18.75-75 mg) could be used, especially for children, in whom losartan is not yet approved by the FDA. A typical dose during the first 48 hours might be: 12.5 mg losartan after exposure to ricin; 12.5 mg-25 mg 12 hours later; 25 mg-50 mg 12 hours later; 50 mg 12 hours later.

### Example 13

Avian influenza A consists of a variety of strains of virus such as H₅N1, H9, H7 and other variants. H stands for hemagglutinin, of which there are 15 subtypes, and N stands for neuraminidase, of which there are 9 subtypes (Kida, H. 2003; PMID: 14619423). H and N are immunogenic surface proteins which define serotypes of the virus.

Influenza A from birds can infect pigs, along with human strains. Pigs thus form a putative “mixing vessel” (Kida, H. 2003; PMID: 14619423), allowing for recombination events to occur. Avian and human strains can recombine, forming viral strains with special virulence for humans. This is thought to be the origin of the 1918 flu pandemic which killed 15-20 million people around the world.

At autopsy, influenza A kills fowl as well as humans primarily by an ARDS-like mechanism (ARDS=acute respiratory distress syndrome; ref. Perkins, Swayne; PMID 12860072; 11924603; 14575094; 11280371). In birds, evidence of inflammation can also be observed in other organs, such as pancreatitis, myocarditis, and meningoencephalitis. Virus can be found replicating in endothelial cells, which may induce a DIC-like picture. The H₅N1 strain of avian influenza kills chickens within 24 hours, consistent with endothelial necrosis and DIC. In this respect, avian influenza is tropic for endothelial cells, like West Nile virus.

Experimentally, the most virulent strains produced necrosis of respiratory epithelium in innoculated mice (PMID: 10627555). Less virulent strains were associated with a robust TNF-alpha response. There was also species variability in response. Gulls were not affected by H5N1 virus, whereas chickens were rapidly killed. This argues for differences in host apoptotic signalling and the innate immune response, not for differences in viral replicative rate. The virus replicates equally well in all species, but some species are asymptomatic.

Viral replication does not necessarily equate to disease; host immune reactivity plays a critical role (PMID: 14766388; 12648460; 9557672; 8811346; 14694127; 14633609; 14615709; 14573812). A reasonable treatment goal for currently untreatable viral diseases would be to convert an otherwise lethal viral infection into asymptomatic viral shedding by the host for 1-2 weeks.

Necrosis and apoptosis of respiratory epithelial cells and endothelial cells is promoted by angiotensin II. Angiotensin II also stimulates an inflammatory response, primarily via type 1 (AT1) receptors. Blocking AT1 receptors may diminish ARDS (see earlier provisional patent applications by GenoMed). Here we propose to use ARBs (angiotensin II type 1 receptor blockers) to treat avian influenza in all vertebrate species, including humans.

The recommended dose of losartan is 0.5-1 mg/kg/day given orally once or twice a day. Other ARBs would be dosed at an equivalent amount. These include irbesartan, telmisartan, candesartan, valsartan, eprosartan, olmesartan. New AT1 receptor antagonists could also be used in this way.

If the flock can be cured of avian flu, there would be no reason to slaughter it in the event that avian flu is detected in the flock. Since a similar treatment should work for humans, and since killed, cooked poultry is non-infectious for humans, there would be no public health reason to kill infected poultry. Since the flock could all recover, there would be no danger from spreading the infection. ARBs could also be administered prophylactically to flocks without any effect on their consummability by humans.

### Example 14

Angiotensin II Inhibition to Treat Osteoporosis

Osteoclasts are derived from macrophages. Activated macrophages express angiotensin I converting enzyme (ACE) on their plasma membrane. Angiotensin II stimulates protein kinase C. Protein kinase C (PKC) stimulates osteoclast function, and resorption of bone (1,2). Osteoporosis is characterized by an imbalance between (exaggerated) bone resoroption and (inadequate) formation.

It is likely that patients with osteoporosis will prove to have an excess frequency of the ACE deletion/deletion (D/D) genotype. Thus, their osteoclasts will generate excessive angiotensin II, and PKC within these cells will undergo excesive autocrine and paracrine stimulation through, presumably, the angiotensin II type 1 receptor.

A treatment for osteoporosis is therefore proposed: the use of a “sartan” or angiotensin II type 1 receptor blocker (“ARB”) at a dose insufficient to lower blood pressure below 100 mmHg systolic. For example, half of a 75 mg dose of AVAPRO (irbesartan) could be used at bedtime in a frail elderly woman with a resting systemic blood pressure of 100/60.

For patients with osteoporosis and hypertension, a hydrophobic ACE inhibitor (e.g. quinapril at ≧2 mg/kg/day in 2 divided doses) would suffice.

### REFERENCES

1) Am J Physiol 1992 July;263 (1 Pt 1): C130-9. Protein kinase C affects microfilaments, bone resorption, and [Ca+]o sensing in cultured osteoclasts. Teti A, Colucci S, Grano M, Argetino L, Zamonin Zallone A. Institute of Human Anatomy, School of Pharmacy, University of Bari, Italy.
2) Bone 2003 March;32(3):217-27. Participation of protein kinase c beta in osteoclast differentiation and function. Lee, S W, Kwak H V, Chung W J, Cheong H, Kim H H, Lee Z H. National Research Laboratory for Bone Metabolism, Chosun University, 501-759, Gwangju, South Korea.

### Example 15

Treatment of common RNA viruses, including monkeypox, which is endemic in West Africa, but which caused a recent outbreak in the Midwest; West Nile virus, which has plagued New York since 1999 and the Midwest since 2001; and the human immunodeficiency virus, or HIV. Monkeypox and West Nile virus have acute mortality rates up to 10%; with current anti-viral thereapy, acute mortality from HIV has been greatly suppressed.

The treatment uses a non-conventional approach: to safely modulate the host's immune response. This approach can obviously be used where no antiviral therapy or vaccine yet exists, as for monkeypox, West Nile virus, and SARS. It can also complement antiviral therapy which may be toxic or expensive, as in HIV, or to cover individuals who failed to be vaccinated.

What all these diverse viruses appear to share is a dependence on angiotensin II for their disease severity. Flu-like symptoms (high fever, chills, muscle aches) come from two major cytokines: tumor necrosis factor-alpha, produced primarily by virally-infected macrophages, and interferon-gamma, made largely by virally-infected T lymphocytes (“T cells”). Severe complications, such as encephalitis in West Nile virus, or respiratory failure in SARS, appear to be caused by an over-exuberant immune response, not by the virus itself. T cells are involved primarily in West Nile virus, and macrophages in SARS.

Angiotensin I-converting enzyme (ACE) is expressed on the surface of activated macrophages and T cells. Since ACE makes angiotensin II, and important immune stimulator, a logical way to try to down-grade the immune response is to use an ACE inhibitor, or an angiotensin II receptor blocker. These two classes of drugs have been used quite safely in hundreds of millions of patients for many years.

Depending on their blood pressure, patients would be prescribed either a hydrophobic ACE inhibitor or an angiotensin II receptor blocker (ARB, also called “-sartan”) by their physician, and their clinical course will be observed by their physician. The expected outcome is that severity of disease will be reduced among volunteers relative to non-participants.

### Example 16

A Method to Avoid Hypersensitivity Reactions

Vaccination occasionally produces hypersensitivity reactions. For example, the recent vaccination of combat troops with the anthrax vaccine has resulted in a number of unexplained cases of “pneumonia” characterized by cough, chest pain, bronchitis, respiratory insufficiency and even death.

The incidence of such reactions may be as high as 20% (Mark Benjamin, UPI, “Vaccine link raised in US troops' deaths”, Aug. 5, 2003), or as low as 602/400,000=0.2% (refs. 1-4).

Hypersensitivity pneumonitis is typically treated with immunosuppression, e.g. systemic corticosteroids. The problem with immunosuppressing a patient with suspected pneumonia is that clinicians are reluctant to use trong, systemic immunosuppression in a situation in which the patient's decline may be due to an unrecognized or inadequately treated micro-organism. Strong immunosuppression under these cicumstances leads to rapid death from disseminated infection.

A method of mild immunosuppression is therefore requried for safe, efficient treatment of patients with possible hypersensitivity reactions, such as pneumonitis, Guillain-Barre syndrome, etc. In addition, mild immunosuppression might decrease the incidence of hypersensitivity reactions to vaccines in the first place.

Hypersensitivity represents an autoimmune attack, i.e. a failure of immune tolerance. Mechanisms of hypersensitivity reactions to vaccines involve antibody (IgM), immune complex (IgM+tissue antigens cross-reactive with the antigen(s) in the vaccine preparation), or T lymphocyte overactivity. Antibodies coat cells in tissues (“opsonization”). Phagocytes bind to antibody-coated cells via their Fc receptors, and engulf the opsonized cells. Tissue inflammation is then further amplified. The classic phagocyte is the macrophage. Immune complexes stimulate compoement activation and phagocytosis by macrophages via a similar mechanism.

Both macrophages and T cells express angiotensin I converting enzyme (ACE) on their surface membrane when activated. The product of ACE, angiotensin II, appears to act in an autocirne and paracrine fashion to activate macrophages and T cells. In other words, antiotensin II is an under-appreciated cytokine. It has been found that an ARB can halt active alopecia greata, and an ACE inhibitor can decrease the intensity of psoriasis, both of which are classic autoimmune diseases.

Therefore, the use of an angiotensin II receptor blocker (ARB) to be given at the time of vaccination to lower the risk of hypersensitivity reactions, and as treatment for patents with established hypersensitivity reactions, such as pneumonitis or back pain in the case of anthrax vaccination (Mark Benjamin, op. cit.) is proposed. A typical dose of an ARB might be 75 mg irbesartan po qhs or similar starting dose of any of the other ARBs. Higher doses could be used for people with hyertension. For patients with significant hypertension, treatment with a hydrophobic ACE inhibitor such a ramipril, quinapril, or trandolapril ought to be effective, although ACE inhibitors produce a mild cough in up to 10% of patients.

### REFERENCES

1) Schuyler M. Link between anthrax immunization and hypersensitivity pneumonitis? Chest. 2003 May;123(5):1769; author reply 1769-1770. PMID: 12740301.
2) Timmer S J, Amundson D E, Malone J D. Hypersensitivity pneumonitis following anthrax vaccination. Chest. 2002 August; 122(2):741-5. PMID: 12171861.
3) Sever J L, Brenner A I, Gale A D, Lyle J M, Moulton L H, West D J; Anthrax Vaccine Export Committee. Safety of anthrax vaccine: a review by the Anthrax Adverse Event Reporting Committee (AVEC) of adverse events reported to the Vaccine Adverse Event Reporting System (VAERS). Pharmacoepidemiol Drug Saf. 2002 April-May;11(3):189-202. PMID: 12051118
4) Halsey N A. Anthrax vaccine and causality assessment from individual case reports. Pharmacoepidemiol Drug Saf. 2002 April-May;11(3):185-7; discussion 2003-4. PMID: 12051117

What is claimed is:

1. A method for treating neuropsychiatric disease comprising administering to a patient in need thereof an effective amount of an angiotensin receptor blocker (ARB).

2. The method according to claim 1 wherein said neuropsychiatric disease, wherein said neuropsychiatric disease is selected from the group consisting of autism, attention deficit hyperactivity disorder, bipolar affective disorder, schizophrenia, and seizure disorder.

3. A process for treating neuropsychiatric disease according to claim 1 wherein said angiotensin receptor blocker is selected from the group consisting of irbesartan, candesartan, losartan, telmisartan, eprosartan and valsartan.

4. A process for treating neuromuscular disease comprising administering to a patient in need thereof an effective amount of an angiotensin receptor blocker.

5. The method of claim 4 wherein said neuromuscular disease is selected from the group consisting of, spinal muscular atrophy syndrome, amyotrophic lateral sclerosis, and administering to said patient an effective amount of angiotensin I receptor blocker.

6. A method for treatment of viral infections comprising administering to a patient in need thereof an effective amount of an angiotensin receptor blocker.

7. The method according to claim 6 wherein the disease is selected from the group consisting of the common cold, respiratory syncytial virus, influenza, hantavirus, SARS, allergic sinusitis, chronic fatigue syndrome, West Nile Virus encephalitis, avian flu and Ebola.

8. A method for treating ricin poisoning comprising administering to a patient in need an effective amount of an angiotensin receptor blocker.

9. The method of claim 8 wherein said ARB is administered immediately after exposure to ricin.

10. A method of treating autoimmune disease comprising administering to a patient in need thereof an effective amount of an ARB.

11. The method of claim 10 wherein the autoimmune disease is selected from the group consisting of psoriasis, alopecia greata, torticollis, vitilligo, rheumatoid arthritis and multiple sclerosis.

12. A method for treating neurodegenerative disease comprising administering to a patient in need thereof an effective amount of an ARB.

13. The method of claim 12 wherein said neurodegenerative disease is selected from the group consisting of spinal muscular atrophy syndromes (types I and II), and amyotrophic lateral sclerosis (ALS).

14. A method for treating a disease associated with excess angiotensin converting enzyme comprising admininstering an effective amount of an ARB to a patient diagnosed with said disease.

15. A method for treating a disease associated with excess angiotensin converting enzyme comprising administering an effective amount of an ARB to a patient having symptoms of said disease.
